(19) United States
(12) Patent Application Publication
(10) Pub. No.: US 2006/0143726 A1
(43) Pub. Date: Jun. 29, 2006

(54) Hydroxypyruvate reductase promoter elements and methods of use thereof in plants

(76) Inventors: Yafan Huang, Ontario (CA); Yang Wang, Ontario (CA)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/534,780

(22) PCT Filed: Nov. 18, 2003
(86) PCT No.: PCT/IB03/06390

§ 371 Date: Oct. 20, 2005

Related U.S. Application Data
(60) Provisional application No. 60/427,204, filed on Nov. 18, 2002.

(51) Int. Cl. A01H 1/00; C12N 5/04; C12N 15/82

(52) U.S. Cl. 800/278; 435/468; 435/419

(57) ABSTRACT

The invention provides compositions and methods for regulating the expression of nucleotide sequence of interest in a plant. Compositions include a novel nucleotide sequence comprising a promoter for the hydroxypyruvate reductase gene. Methods for expressing a nucleotide sequence of interest in a plant using the promoter sequences disclosed herein is provided. The method comprises stably incorporating into the genome of a plant cell a nucleotide sequence operably linked to the promoter of the present invention and regenerating a stably transformed plant that expresses the nucleodide sequence.

### FIELD OF THE INVENTION

The invention relates to the field of plant genetic engineering. More particularly, the invention relates to a hydroxypyuvate reductase promoter. The invention also relates to heterologous nucleic acid constructs, vectors, transformation methods, and transgenic plant cells, transgenic plants, and transgenic seeds.

### BACKGROUND OF THE INVENTION

Transcriptional gene regulation is a key component to the success of any genetic manipulation. Promoters are genetic elements that bind the transcriptional apparatus and typically initiate the transcription of a downstream gene or sequence. The downstream gene or sequence can be a selectable marker, a gene encoding a protein of interest, an antisense nucleic acid sequence or a portion of the forgoing or sequences that will form non-translatable mRNA such as hairpin structures.

Promoters useful for genetic engineering can typically be classified as 1) constitutive, providing gene expression in any cell, 2) tissue specific, providing expression in a limited type of cell or during developmental specific time periods, or 3) inducible, providing expression in response to a cellular or extracellular stimulus. While constitutive promoters have found widespread use to date, there are circumstances where it is desirable to restrict or limit expression of the heterologous sequence.

It is therefore desirable to develop plant promoters which have specific expression characteristics and maintain a high level of expression in those tissues or cells.

### SUMMARY OF THE INVENTION

The present invention is based in part upon the discovery of hydroxypyrnvate reductase nucleic acid sequences, polypeptides and promoter sequence isolated from Arabidopsis thaliana. The promoters, nucleic acids, polynucleotides, proteins and polypeptides, or fragments thereof described herein are collectively referred to as HPR promoters, nucleic acids and polypeptides.

Accordingly, in one aspect, the invention provides an isolated HPR promoter nucleic acid molecule that includes the sequence of SEQ ID NO:4 or 5 or a fragment, homolog, analog or derivative thereof. The HPR promoter nucleic acid is less than 1000 nucleotide in length. Preferably, the HPR promoter is less than 800, 750, or 600 nucleotides in length.

The HPR promoter regulates transcription of an operably linked nucleotide sequence of interest. The HPR promoter regulates transcription constitutively. Alternatively transcription is regulated ins response to a stimulus, such as light or an environmental stress (e.g., drought).

Accordingly, in another aspect, the invention provides nucleic acid constructs including an HPR promoter (e.g., SEQ ID NO:4 or 5) fragment, homolog, analog or derivative thereof operably linked to a nucleotide sequence encoding a gene. Alternatively, the HPR promoter is operably linked to a non-translatable mRNA molecule of a gene. A non-translatable mRNA molecule includes for example an antisense nucleic acid, a hairpin RNA or a microRNA. The nucleic acid constructs are referred to herein as HPR promoter-gene constructs. Optionally, HPR promoter-gene constructs include a nucleic acid encoding a selectable marker such as kanamycin or a reporter gene such as GUS.

The gene is heterologous. Alternatively, the gene is heterologous. By heterologous gene it is meant a gene other than the native gene which the HPR promoter regulates, i.e., hydroxypyruvate reductase. The heterologous gene encodes a protein of interest or fragment thereof. A heterologous gene alters an agronomic trait. An agronomic trait is, for example, disease resistance, herbicide resistance, environmental stress resistance, enhanced growth or increased yield. The heterologous gene is a plant gene. Alternatively, the heterologous gene is a non-plant genes, e.g., mammalian, bacterial, or insect gene. The heterologous gene is a structural gene such as an enzyme, (e.g., farnesyl transferase alpha, farnesyl transferase beta or CaaX prenyl protease), a chaperonin protein (e.g., HSP or Ras), a scaffolding protein or a transcriptional regulator.

Also included in the invention are vectors containing one or more of the HPR promoters or HPR promoter-gene constructs described herein, and a cell containing the vectors. The cell is a plant cell. The plant cell is monocotyledonous. Alternatively, the plant cell is dicotyledonous.

The invention is also directed to plants and cells transformed with a promoter or a vector comprising a HPR promoter. Also included in the invention is the seed,and progeny of the transformed plants or cells.

The invention also includes a method of producing a transgenic plant (e.g., monocot or dicot) by introducing to a plant cell a vector containing a HPR promoter-gene construct of the invention to generate a transgenic cell and regenerating a transgenic plant from the transgenic cell. The transgenic plant expresses the protein of interest. Alternatively, the transgenic plant expresses the protein of interest at a decreased level compared to a wild type (non-transformed plant). For example, when a plant cell is transformed with a HPR promoter-gene construct containing a nucleic acid encoding for a gene of interest, the resulting transgenic plant has an increased level of expression compared to a wild-type plant. Similarly, when a plant cell is transformed with a HPR-gene construct containing a nucleic acid encoding a non-translatable mRNA molecule of a gene encoding a protein of interest, the resulting transgenic plant has a decreased level of expression compared to a wild-type plant. In some aspects the transgenic plant has an altered phenotype such as, but not limited to, increased tolerance to stress, altered senescence profile, increased ABA sensitivity, increased yield, increased productivity and increased biomass compared to a wild type plant. Stress can include a wide variety of conditions including, but not limited to, drought, heat, salt, photo-oxidative stress or nutrient deficiency. The invention also includes the transgenic plant, the seed, and progeny of the transformed plants produced by the methods of the invention.

The invention further provides a method for expressing a heterologous protein by providing a cell, e.g., a plant cell containing a HPR promoter-gene construct, a vector that includes a HPR promoter-gene construct, and culturing the cell under conditions sufficient to express the heterologous protein encoded by the nucleic acid. The expressed protein is then recovered from the cell. The plant cell is monocotyledonous. Alternatively, the plant cell is dicotyledonous. Further, a plant cell containing the HPR promoter-gene construct is regenerated to produce a transgenic plant, the transgenic plant expressing the heterologous protein. The heterologous protein may be purified from the plant tissue or alternatively the heterologous protein may have a biological activity and impart a phenotype to the transgenic plant.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1. is a schematic representation of the HPR vector constructs; A) pRD29A-anti-FTA, B) pBI121-HPR, C) pRD29A-HPR, D) pBI121-hp-HPR.

FIG. 2. is a schematic representation of the HPR promoter vector constructs; A) pHPR-GUS, B) pHPRT-GUS.

FIG. 3. is a illustration of control and transgenic Arabidopsis plants stained for GUS activity, Row A contains Wild-type and 35S-GUS transgenic seedlings; Row B contains HPR-GUS and pHPRT-GUS transgenic seedlings and Row C contains pHPR-GUS and pHPRT-GUS transgeneic roots

FIG. 4. is an illustration of GUS activity in control and pHPR-GUS transgenic Brassica napus. Panel A contains a wild type seedling and four transgenic seedlings. Panel B contains siliques and developing embryos from a wild type and transgenic plant.

FIG. 5. is an illustration of GUS activity in controls and transformed corn callus tissues. Row 1 contains corn callus transformed with the pHPR-GUS construct. Well 2A contains corn callus tissue exposed to Agrobacterium lacking the construct and no transformation protocol. Well 2B contains corn callus exposed to Agrobacterium lacking the pHPR-GUS construct and exposed to the transformation protocol. Well 3A contains Agrobacterium culture but no corn callus tissue.

### DETAILED DESCRIPTION OF INVENTION

The present invention provides structural DNA sequences and their promoters, which are active in the tissue of both monocotyledonous and dicotyledonous plants, in particular, the aerial tissue. The present invention is based in part on the discovery of a novel promoter sequence (SEQ ID NO:4), of the hydroxyxpyruvate reductase (HPR) gene isolated from Arabidopsis thaliana (At). Also provided is a truncated variation of the promoter sequence. (SEQ ID NO:5). The promoter sequences-are collectively referred to herein as “HPR promoters”, or “promoters” The HPR promoters produce high levels of gene expression in Arabidopsis, Brassica (canola) and Zea maise (corn). Northern analysis and GUS reporter activity demonstrate expression in the aerial tissues. Expression is inducible in all shoot tissue, with minimal root expression, by light and in response to environmental stress, such as drought stress. The HPR promoters herein are suitable for applications that require strong expression specifically in aerial tissue. Additionally, if desired the HPR promoter can be modified to include some root expression. (SEQ ID NO:5)

The invention further provides the hydroxpyruvate reductase (HPR) nucleic acid sequence (SEQ ID NO: 1) and the encoded polypeptide: SEQ ID NO:2, isolated from Arabidopsis thaliana (At) and HPR antisense nucleic acids. (SEQ ID NO:3). The sequences are collectively referred to as “HPR nucleic acids”, HPR polynucleotides” or “HPR antisense nucleic acids” and the corresponding encoded polypeptide is referred to as a “HPR polypeptide” or “HPR protein”.

Unless indicated otherwise, “HPR” is meant to refer to any of the sequences diclosed herein. Table A below summarizes the nucleic acids and polypeptides according to the invention

TABLE A

| | | |
|---|---|---|
| SEQ | | |
| ID | NAME | NT Sequence |

| | | |
|---|---|---|
| 1 | HPR NT+ | |
| 2 | HPR AA | |
| 3 | HPR NT− | |
| 4 | HPR Promoter | |
| 5 | HPR Promoter | |
| | Truncated | |
| 6 | HPRClal | AAATCGATATGGCGAAACCGGTGTCC |
| 7 | HPRBamHI | CGGGATCCTCATAGCTTCGAAACAGGCAA |
| 8 | HPRBamFW | AAAGGATCCATGGCGAAACCGGTGTCCAT |
| 9 | RD29AP1 | TTTAAGCTTGGAGCCATAGATGCAATTCAA |
| 10 | RD29AP2 | AAATCTAGACTTTCCAATAGAAGTAATCAAACC |
| 11 | HPRXbaRE | AAATCTAGACGTTTCCATGTCACAGGTTG |
| 12 | HPRSacFW | AAAGAGCTCATGGCGAAACCGGTGTCCAT |
| 13 | HPRSacRE | AAAGAGCTCCGTTTCCATGTCACAGGTTG |
| 14 | HPRP1Hind | AAAAAGCTTGAAGCAGCAGAAGCCTTGAT |
| 15 | HPR2Bam | AAAGGATCCCGCCATGGTAGAGAAAAGAGA |
| 16 | HPR3Hind | AAAAAGCTTACGTCAGCATTATCTCGTTAC |
| 17 | Adapter 1 | CTAATACGACTCACTATAGGGCTCGAGCGGC |
| | | CGCCCGGGCAGGT |
| 18 | Adapter 2 | ACCTGCCC-NH2 |
| 19 | AP1 | GGATCCTAATACGACTCACTATAGGGC |
| 20 | 28w1 | AGCTGGCGTAATAGCGAAGA |
| 21 | AP2 | CTATAGGGCTCGAGCGGC |
| 22 | 28w2 | CGTTGGAGTCCACGTTCTTT |
| 23 | 28LAP1 | GTTACTGCTGTGTTTCTTGCGAGGTGACTC |
| 24 | 28LAP2 | CTCAAAGCTGAGAACAGAGTCTCTCCCCAATC |
| 25 | NPT 1 | ATTGCACGCAGGTTCTCCGG |
| 26 | NPT 2 | ATCGGGAGCGGCGATACCG |
| 27 | GG9 | CTGCATCCGGCGACCTTGTTC |
| 28 | HPRXbaFW | AAATCTAGAATGGCGAAACCGGTGTCC |
| 29 | HPRSaIRV | AAAGTCGACTCATAGCTTCGAAACAGGC |

Hydroxypyruvate reductase is a component of the photorespiratory pathway. The photorespiratory pathway is a system which evolves carbon dioxide and consumes oxygen. Ribisco can react with O₂ resulting in synthesis of 3-phosphoglycerate (3-PGA) and 2-phosphoglycolate. Two molecules of 2-phosphoglycolate are converted to one CO₂ and one molecule of 3-PGA thereby salvaging 3 molecules of carbon at the cost of one molecule of carbon as CO₂. The pathways of phosphoglycolate metabolism involves three organelles, chloroplast, peroxisome and mitochondrion and the integration of photorespiratory carbon and nitrogen cycles. HPR is localized exclusively in the peroxisome and functions in the last step of photorespiration pathway, converting hydroxypyruvate to glycerate and NADH to NAD. The glycerate is shuttled into chloroplast, and subsequently converted into glycerate-3-phosphate, which enters the Benson-Calvin cycle for production of carbon skeletons. Recent isotope-labeled feeding studies have firmly established that chloroplast is the site for ABA biosynthesis, and that pyrnvate and glyceraldehyde 3-phosphate are the ultimate precusors of ABA. One of these experiments in which pyruvate and other carbohydrate molecules were incubated with intact spinach chloroplasts confirms that pyruvate is incorporated more efficiently into ABA than other compounds such as mevalonic acid (MVA). It is now known that the first step of ABA biosynthesis is the formation of the C₅ isoprene isopentenyl diphosphate (IPP) from pyruvate and glyceraldyhyde 3-phsophate. The C₅ isoprene is then converted to β-carotene which is subsequently converted to ABA through multiple steps.

The integration of the organelles and the shuttling of intermediates between them provides for the glycerate, formed in the peroxisome, to be transported to the chloroplast. In the chloroplast, conversion to 3-phosphoglycerate occurs by glycerate kinase. The fate of the 3-phosphoglycerate will be determined by the affinity of various enzymes for this substrate. For example, chloroplasts also contain a glycolytic pathway catalyzed by plastid specific glycolytic enzymes. This pathway provides a means of converting the 3-phosphoglycerate to pyruvate for ABA biosynthesis, also a chloroplast localized process. Hydroxypyruvate reductase activity and mRNA transcription is regulated in part by the plant hormone cytokinin and light. (Greenler et al. 1989) Cytokinins are a group of plant hormones that have influence on numerous cellular processes such as cell division, chloroplast development, cell differentiation and some enzyme activity. HPR expression and activity are detectable in both light and dark grown seedlings however, both are induced in cotyledon (Hondred et al. 1987) and leaf (Greenler and Becker 1990) in a light dependent manner.

Plant cDNAs have been identified encoding the HPR gene from cucumber (Greenler et al. 1989) and pumpkin (Hayashi et al. 1996). The promoter adjacent to the cucumber gene has also been isolated and characterized by deletion analysis (Sloan et al. 1993, Daniel and Becker 1995). The cucumber HPR promoter was analyzed in transgenic tobacco and was determined to require at least 299 bp of 5′-HPR-flanking region to produce high-level light-regulated expression (Sloan et al. 1993). Continued analysis identified the regions necessary for light regulation, leaf-specific expression and a negative root element responsible for silencing root expression (Daniel and Becker 1995). In addition, a cytokinin response element from the cucumber promoter has also been mapped (Jin et al. 1998). In a BLAST analysis, it was determined that the cucumber HPR promoter shares no sequence homology, with the HPR promoter of the present invention that was isolated from Arabidopsis. Specifically, the Arabidopsis sequence is half the length of the corresponding cucumber sequence.

In a BLAST search of public sequence databases, it was found that the disclosed 1161 nucleotide Arabidopsis thaliana HRP nucleic acid sequence (SEQ ID NO: 1) which encodes a HPR amino acid sequence (SEQ ID NO;2) is 99.8% identical to a portion of the 1428 nucleotide Arabidopsis thaliana hydroxypyruvate reductase sequence of Genbank Accession Number D85339 and is identical to a portion of the nucleotide Arabidopsis thaliana HRP sequence of Genbank Accession Number AC012563.

The compositions of the invention are useful in regulating the expression of an operably linked nucleotide sequence of interest. The present invention further provides nucleotide constructs that allow initiation of transcription in both a constitutive and an inducible manner. For example, transcription of the operably linked nucleotide sequence is initiated or increased in the presence of a stimulus. A stimulus includes, light, a pathogen, tissue wounding, such as wounding resulting from insect herbivory, leaf breakage by physical means, hormone or chemical exposure, particularly hormones or chemicals associated with wounding (i.e., wound-responsive chemicals) or an environmental stress such as chilling stress, salt stress, and water stress.

Additional utilities for HPR promoters, HPR nucleic acids and polypeptides according to the invention are disclosed herein.

HPR Promoters and Promoter Constructs

The invention provides previously unidentified promoter nucleic acid sequences isolated from the Arabidopsis thaliana (At) HPR gene. The term “promoter” refers to a region of DNA upstream from the translational start codon which is involved in recognition and binding of RNA polymerase and other proteins to initiate transcription.

The HPR promoter sequence of the invention includes the nucleic acid sequence of SEQ ID NO:4. The HPR promoter sequences of the invention are typically identical to or show substantial sequence identity nucleic acid sequence depicted in SEQ ID NO:4. or fragments thereof. A HPR promoter sequence is at least 25%, 50%, 75%, 80%, 85%, 90%, 95%, 96%, 97%, 98% 99 identical to SEQ ID NO: 4.

The HPR promoter sequence is 512 nucleotides in length. All or part (i.e., fragment) of the HPR promoter may be used to specifically direct expression of a sequence or gene to plant tissue. Optionally, the HPR promoter contains additional nucleic acid sequences at the 5′ or 3′ end. The additional nucleic acid sequence is a coding sequence. Alternatively, the additional nucleic acid sequence is a non-coding sequence. For example, the promoter includes other transcriptional and translational regulatory nucleic acid sequences (also termed “control sequences”) that are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences. A HPR promoter sequence is less than 1000 nucleotides in length, e.g., less than or equal to 800, 750, 600, 625, 600, 550; 525 nucleotides in length. In various aspects, the HPR promoter includes the nucleic acid sequence of one or more of SEQ ID NOs: 4 or 5.

The HPR promoters are capable of conferring high levels (i.e., strong promoter) of transcription in plant tissue when used as a promoter for a heterologous coding sequence. As used herein, “promoter strength” refers to the level of promoter-regulated expression of a heterologous gene in a plant tissue or tissues, relative to a standard (a standard gene promoter, e.g., the 35S CaMV promoter or the CsVMV promoter). Expression levels is measured by linking the promoter to a suitable reporter gene such as GUS (beta.-glucuronidase). Expression of the reporter gene can be easily measured by fluorometric, spectrophotometric or histochemical assays.

Various modifications are made to the promoters of the invention to provide promoters with different properties (e.g., tissue specificity, promoter strength, and the like). For example, truncated forms of a HPR promoter are constructed by mapping restriction enzyme sites in the promoter and then using the constructed map to determine appropriate restriction enzyme cleavage to excise a subset of the sequence. The modified promoters are then inserted into a suitable vector and tested for their ability to drive expression of a marker gene. Tissue specificity of the modified promoters is tested in regenerated plants. An exemplary modified HPR promoter includes the nucleic acid of SEQ ID NO:5 which allows for expression of a gene of interest in the root tissue of a plant.

HPR promoters are isolated in a variety of ways know in the art. For example, HPR promoters are isolated from genomic DNA fragments encoding a HPR protein and which also contain sequences upstream from the sequence encoding the HPR protein. Genomic fragments encoding HPR proteins are isolated by methods known in the art. HPR promoter sequences are isolated by screening plant DNA libraries with oligonucleotide probes having sequences derived from the DNA sequence of the HPR promoter of SEQ ID NO:4. Other methods known to those of skill in the art can also be used to isolate plant DNA fragments containing HPR promoters. See Sambrook, et al. for a description of other techniques for the isolation of DNAs related to DNA molecules of known sequence. For instance, deletion analysis and a promoterless reporter gene (e.g., GUS) can be used to identify those regions which can drive expression of a structural gene. Sequences characteristic of promoter sequences can also be used to identify the promoter. Sequences controlling eukaryotic gene expression have been extensively studied. For instance, promoter sequence elements include the TATA box consensus sequence (TATAAT), which is usually 20 to 30 base pairs upstream of the transcription start site. In most instances the TATA box is required for accurate transcription initiation. In plants, further upstream from the TATA box, at positions −80 to −100, there is typically a promoter element with a series of adenines surrounding the trinucleotide G (or T) N G. J. Messing et al., in Genetic Engineering in Plants, pp. 221-227 (Kosage, Meredith and Hollaender, eds. 1983).

The HPR promoter is useful in ligating or fusing (i.e., operably linked) to the 5′ end of one or more nucleic acid sequences (e.g., gene) thereby producing a HPR promoter--gene construct. The term “operably linked” as used herein refers-to linkage of a promoter upstream from a DNA sequence such that the promoter mediates transcription of the DNA sequence. The HPR promoter is ligated in frame upstream of a sequence to be expressed. Downstream or 3′ of the sequence to be expressed may be suitable transcription termination signals, including a polyadenylation signal or other sequences found helpful in the processing of the 3′ mRNA terminus. The promoter sequence also includes transcribed sequences between the transcriptional start and the translational start codon. Optionally, the construct contains a nucleic acid encoding a reporter gene or a selectable marker ligated 3′ of the HPR promoter. The reporter/marker sequence provides a means to easily identify the cells expressing the sequences under control of the HPR promoter. For example, selectable marker genes encode a polypeptide that permits selection of transformed plant cells containing the gene by rendering the cells resistant to an amount of an antibiotic that would be toxic to non-transformed plant cells. Selectable marker genes include the neomycin phosphotransferase (nptII) resistance gene, hygromycin phosphotransferase (hpt), bromoxynil-specific nitrilase (bxn), phosphinothricin acetyltransferase enzyme (B3AR) and the spectinomycin resistance gene (spt), wherein the selective agent is kanamycin, hygromycin, geneticin, the herbicide glufosinate-ammonium (“Basta”) or spectinomycin, respectively.

The nucleic acid sequences is heterologous (i.e., exogenous) to the promoter. Exogenous and heterologous, as used herein, denote a nucleic acid sequence which is not obtained from and would not normally form a part of the genetic make-up of the plant or the cell to be transformed, in its untransformed state. Foreign genes and sequences, for purposes of the present invention, are those which are not naturally occurring in the plant into which they are delivered. Portions of the above mentioned heterologous or exogenous sequences and foreign genes and sequences are of plant origin, however, the HPR promoter-gene construct forms a combination or variant not naturally occurring in the plant The nucleic acid encodes for a protein of interest or fragment thereof. The gene is for example a structural gene, an enzyme (e.g., farnesyl transferase, alpha or beta or CaaX prenyl protease), a chaperonin protein (e.g., HSP or Ras)), a scaffolding protein, or a transcriptional regulator. For example, the nucleic acid encodes for a gene capable of altering an agronomic trait such as disease resistance, herbicide resistance, environmental stress resistance or increased yield. Alteration of prenylation by increasing or decreasing farnesyl transferase, CaaX prenyl protease activity has been shown to elicit plants with altered agronomic traits. (See for example, PCT US 98/15664, U.S. Ser. No. 03/26894, WO 02/097097 and WO 03/012116, each of which are incorporated by reference in their entireties)

The nucleic acid sequences are DNA, such as cDNA and genomic DNA or RNA, such as mRNA and tRNA. For example, the nucleic acid sequence is a non-translated mRNA molecule of a gene or fragment thereof that encodes a protein of interest. Non-translated mRNA includes, e.g., antisense, hairpin RNA, microRNA, or ribozymes. The non-translated mRNA may alter agronomic traits, including those identified above. Alternatively, the non-translated mRNA may prevent the translation of sequences which are detrimental to the plant.

The HPR promoter-gene contains one promoter nucleic acid sequence. Alternatively, the HPR promoter-gene construct contains 2, 3, 4, 5, or more promoter nucleic acid sequences. Optionally, the promoter sequences are linked together by a spacer. No particular length is implied by the term spacer. The spacer is less than 1000 nucleotides in length, e.g., less than or equal to 900, 800, 700, 500, 250, 100, 75, 50, 35, 25, or 10 nucleotides in length.

The promoter regulates expression of the nucleic acid sequence of interest constitutively. Alternatively, the promoter is inducible by a stimulus such as light or an environmental stress, such as drought, chilling stress, salt stress, a pathogen, a herbicide, or wounding. The terms “constitutive promoter ” as used herein refer to a promoter which is capable of expressing operably linked DNA sequences in all tissues or nearly all tissues of a plant. The terms “inducible promoter”, as used herein, refer to plant promoters that are capable of selectively expressing operably linked DNA sequences at particular times in response to endogenous or external stimuli.

Also included in the invention are vectors containing the HPR promoter-gene constructs. Suitable plant expression vectors systems include tumor inducing (Ti) plasmid or portion thereof found in Agrobacterium, cauliflower mosaic virus (CaMV) DNA and vectors such as pBI121 .

For expression of HPR promoter gene construct in plants, the recombinant expression cassette will contain in addition to the HPR promoter and nucleic acid of interest, a transcription initiation site (if the coding sequence to transcribed lacks one), and a transcription termination/polyadenylation sequence. The termination/polyadenylation region may be obtained from the same gene as the promoter sequence or may be obtained from different genes. Unique restriction enzyme sites at the 5′ and 3′ ends of the cassette are typically included to allow for easy insertion into a pre-existing vector.

Additional regulatory elements that may be connected to the HPR promoter gene construct for expression in plant cells include terminators, polyadenylation sequences, and nucleic acid sequences encoding signal peptides that permit localization within a plant cell or secretion of the protein from the cell. Plant signal sequences, including, but not limited to, signal-peptide encoding DNA/RNA sequences which target proteins to the extracellular matrix of the plant cell (Dratewka-Kos, et al., J. Biol. Chem., 264: 4896-4900 (1989)) and the Nicotiana plumbaginifolia extension gene (DeLoose, et al., Gene, 99: 95-100 (1991)), or signal peptides which target proteins to the vacuole like the sweet potato sporamin gene (Matsuka, et al., Proc. Nat'l Acad. Sci. (USA), 88: 834 (1991)) and the barley lectin gene (Wilkins, et al., Plant Cell, 2: 301-313 (1990)), or signals which cause proteins to be secreted such as that of PRIb (Lind, et al., Plant Mol. Biol., 18: 47-53 (1992)), or those which target proteins to the plastids such as that of rapeseed enoyl-ACP reductase (Verwaert, et al., Plant Mol. Biol., 26: 189-202 (1994)) are useful in the invention.

A number of types of cells may act as suitable host cells for expression of the vectors. Plant host cells include cells from monocots and dicots. For example, plant cells include epidermal cells, mesophyll and other ground tissues, and vascular tissues in leaves, stems, floral organs, and roots from a variety of plant species, such as Arabidopsis thlaliana, Nicotiana tabacum, Brassica napus, Zea mays, Oryza sativa, Gossypium hirsutum and Glycine max.

HPR Nucleic Acids

The nucleic acids of the invention include those that encode a HPR polypeptide or protein. As used herein, the terms polypeptide and protein are interchangeable.

In some embodiments, a HPR nucleic acid encodes a mature HPR polypeptide. As used herein, a “mature” form of a polypeptide or protein described herein relates to the product of a naturally occurring polypeptide or precursor form or proprotein. The naturally occurring polypeptide, precursor proprotein includes, by way of nonlimiting example, the full length gene product, encoded by the corresponding gene. Alternatively, it may be defined as the polypeptide, precursor proprotein encoded by an open reading frame described herein. The product “mature” form arises, again by way of nonlimiting example, as a result of one or more naturally occurring processing steps that may take place within the cell in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

Among the HPR nucleic acids is the nucleic acid whose sequence is provided in SEQ ID NO:1 or a fragment thereof. Additionally, the invention includes mutant or variant nucleic acids of SEQ ID NO:1 or a fragment thereof, any of whose bases may be changed from the corresponding base shown in SEQ ID NO:1 while still encoding a protein that maintains at least one of its HPR-like activities and physiological functions. The invention further includes the complement of the nucleic acid sequence of SEQ ID NO:1 including fragments, derivatives, analogs and homologs thereof. Complement nucleic acid HPR sequences include SEQ ID NO:3. The invention additionally includes nucleic acids or nucleic acid fragments, or complements thereto, whose structures include chemical modifications.

One aspect of the invention pertains to isolated nucleic acid molecules that encode HPR proteins or biologically active portions thereof Also included are nucleic acid fragments sufficient for use as hybridization probes to identify HPR-encoding nucleic acids (e.g., HPR mRNA) and fragments for use as polymerase chain reaction (PCR) primers for the amplification or mutation of HPR nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated HPR nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO:1, a complement of any of this nucleotide sequence, can be isolated using standard molecular biology techniques and the sequence information provided herein. Using all or a portion of the nucleic acid sequence of SEQ ID NO:1 as a hybridization probe, HPR nucleic acid sequences or its promoter can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to HPR nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of SEQ ID NO:1 or SEQ ID NO:4 or a complement thereof Oligonucleotides may be chemically synthesized and may be used as probes.

In another embodiment, an isolated nucleic acid molecule of the invention includes a nucleic acid molecule that is a complement of the nucleotide sequence shown in SEQ ID NO:1. In another embodiment, an isolated nucleic acid molecule of the invention comprises a nucleic acid molecule that is a complement of the nucleotide sequence shown in SEQ ID NO:1 or a portion of these nucleotide sequence. A nucleic acid molecule that is complementary to the nucleotide sequence shown in SEQ ID NO:1 is one that is sufficiently complementary to the nucleotide sequence shown in SEQ ID NO:1 that it can hydrogen bond with little or no mismatches to the nucleotide sequence shown in SEQ ID NO:1, thereby forming a stable duplex. Exemplary complement nucleic acid sequences to SEQ ID NO:1 include the sequences of SEQ ID NO:3.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of SEQ ID NO:1 e.g., a fragment that can be used as a probe or primer, or a fragment encoding a biologically active portion of HPR or a natural promoter of HPR. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which is incorporated herein by reference in its entirety). A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a HPR polypeptide. Isoforms can be expressed in different tissues of the same organism as a result of, for example, alternative splicing of RNA. Alternatively, isoforms can be encoded by different genes. Homologous nucleotide sequences also include, but are not limited to, naturally occurring allelic variations and mutations of the nucleotide sequences set forth herein. Homologous nucleic acid sequences include those nucleic acid sequences that encode conservative amino acid substitutions (see below) in SEQ ID NO:2 as well as a polypeptide having HPR activity, e.g. substrate binding.

The nucleotide sequence determined from the cloning of the Arabidopsis thaliana HPR gene allows for the generation of probes and primers designed for use in identifying and/or cloning HPR homologues in other cell types, e.g., from other tissues, as well as HPR homologues from other plants. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO:1; or an anti-sense strand nucleotide sequence of SEQ ID NO:1; or of a naturally occurring mutant of SEQ ID NO:1.

Probes based on the Arabidopsis thaliana HPR nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a HPR protein, such as by measuring a level of a HPR-encoding nucleic acid in a sample of cells from a subject e.g., detecting HPR mRNA levels or determining whether a genomic HPR gene has been mutated or deleted.

A “polypeptide having a biologically active portion of HPR” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of HPR” can be prepared by isolating a portion of SEQ ID NO:1 that encodes a polypeptide having a HPR biological activity (biological activities of the HPR proteins are described below), expressing the encoded portion of HPR protein (e.g., by recombinant expression in vitro) and assessing the activity of the encoded portion of HPR. In another embodiment, a nucleic acid fragment encoding a biologically active portion of HPR includes one or more regions.

Antisense HPR Nucleic Acids

Another aspect of the invention pertains to isolated antisense nucleic acid molecules that are hybridizable to or complementary to the nucleic acid molecule comprising the nucleotide sequence of SEQ ID NO:1 or fragments, analogs or derivatives thereof. An “antisense” nucleic acid comprises a nucleotide sequence that is complementary to a “sense” nucleic acid encoding a protein, e.g. complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire HPR coding strand, or to only a portion thereof. Nucleic acid molecules encoding fragments, homologs, derivatives and analogs of a HPR protein of SEQ ID NO:2 or antisense nucleic acids complementary to a HPR nucleic acid sequence of SEQ ID NO:1 are additionally provided. Exemplary HPR anti-sense nucleic acid include the nucleic acid sequences of SEQ ID NO:3.

In one embodiment, an antisense nucleic acid molecule is antisense to a “coding region” of the coding strand of a nucleotide sequence encoding HPR. The term “coding region” refers to the region of the nucleotide sequence comprising codons which are translated into amino acid residues (e.g., the protein coding region of Arabidopsis thaliana HPR corresponds to SEQ ID NO:2 ). In another embodiment, the antisense nucleic acid molecule is antisense to a “noncoding region” of the coding strand of a nucleotide sequence encoding HPR. The term “noncoding region” refers to 5′ and 3′ sequences which flank the coding region that are not translated into amino acids (i.e., also referred to as 5′ and 3′ untranslated regions).

Given the coding strand sequences encoding HPR disclosed herein (e.g. SEQ ID NO:1), antisense nucleic acids of the invention can be designed according to the rules of Watson and Crick or Hoogsteen base pairing. The antisense nucleic acid molecule can be complementary to the entire coding region of HPR mRNA, but more preferably is an oligonucleotide that is antisense to only a portion of the coding or noncoding region of HPR mRNA. For example, the antisense oligonucleotide can be complementary to the region surrounding the translation start site of HPR mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, S-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are generated in situ such that they hybridize with or bind to cellular mRNA and/or genomic DNA encoding a HPR protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol I or pol III promoter are preferred.

In yet another embodiment, the antisense nucleic acid molecule of the invention is an a-anomeric nucleic acid molecule. An ot-anomeric nucleic acid molecule forms specific double-stranded hybrids with complementary RNA in which, contrary to the usual D-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA-DNA analogue (Inoue et al. (1987) FEBS Lett 215: 327-330).

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in applications.

Double Stranded RNA Inhibition (RNAi) by Hairpin Nucleic Acids

Another aspect of the invention pertains to the use of post transcriptional gene silencing (PTGS) to repress gene expression. Double stranded RNA can initiate the sequence specific repression of gene expression in plants and animals. Double stranded RNA is processed to short duplex oligomers of 21-23 nucleotides in length. These small interfering RNA's suppress the expression of endogenous and heterologous genes in a sequence specific manner (Fire et al. Nature 391:806-811, Carthew, Curr. Opin. in Cell Biol., 13:244-248, Elbashir et al., Nature 411:494-498). A RNAi suppressmg construct can be designed in a number of ways, for example, transcription of a inverted repeat which can form a long hair pin molecule, inverted repeats separated by a spacer sequence that could be an unrelated sequence such as GUS or an intron sequence. Transcription of sense and antisense strands by opposing promoters or cotranscription of sense and antisense genes.

HPR Ribozymes and PNA Moieties

In still another embodiment, an antisense nucleic acid of the invention is a ribozyme. Ribozymes are catalytic RNA molecules with ribonuclease activity that are capable of cleaving a single-stranded nucleic acid, such as a mRNA, to which they have a complementary region. Thus, ribozymes (e.g., hammerhead ribozymes (described in Haselhoff and Gerlach (1988) Nature 334:585-591)) can be used to catalytically cleave HPR mRNA transcripts to thereby inhibit translation of HPR mRNA. A ribozyme having specificity for a HPR-encoding nucleic acid can be designed based upon the nucleotide sequence of a HPR DNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a HPR-encoding mRNA. See, e.g., Cech et al. U.S. Pat. No. 4,987,071; and Cech et al. U.S. Pat. No. 5,116,742. Alternatively, HPR mRNA can be used to select a catalytic RNA having a specific ribonuclease activity from a pool of RNA molecules. See, e.g., Bartel et al., (1993) Science 261:1411-1418.

Alternatively, HPR gene expression can be inhibited by targeting nucleotide sequences complementary to the regulatory region of the HPR (e.g., the HPR promoter and/or enhancers) to form triple helical structures that prevent transcription of the HPR gene in target cells. See generally, Helene. (1991) Anticancer Drug Des. 6: 569-84; Helene. et al. (1992) Ann. N.Y. Acad. Sci. 660:27-36; and Maher (1992) Bioassays 14: 807-15.

In various embodiments, the nucleic acids of HPR can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of HPR can be used in therapeutic and diagnostic applications. For example, PNAs can be used as antisense or antigene agents for sequence-specific modulation of gene expression by, e.g., inducing transcription or translation arrest or inhibiting replication. PNAs of HPR can also be used, e.g., in the analysis of single base pair mutations in a gene by, e.g., PNA directed PCR clamping; as artificial restriction enzymes when used in combination with other enzymes, e.g., SI nucleases (Hyrup B. (1996) above); or as probes or primers for DNA sequence and hybridization (Hyrup et al. (1996), above; Perry-O'Keefe (1996), above).

In another embodiment, PNAs of HPR can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of HPR can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation ([yrup (1996) above). The synthesis of PNA-DNA chimeras can be performed as described in Hyrup (1996) above and Finn et al. (1996) Nuc Acids Res 24: 3357-63. For example, a DNA chain can be synthesized on a solid support using standard phosphoramidite coupling chemistry, and modified nucleoside analogs, e.g., 5′-(4-methoxytrityl) amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

HPR Polypeptides

A HPR polypeptide of the invention includes the protein whose sequence is provided in SEQ ID NO:2. The invention also includes a mutant or variant protein any of whose residues may be changed from the corresponding residue shown in SEQ ID NO:2 while still encoding a protein that maintains its HPR-like activities and physiological functions, or a functional fragment thereof. In some embodiments, up to 20% or more of the residues may be so changed in the mutant or variant protein. In some embodiments, the HPR polypeptide according to the invention is a mature polypeptide.

In general, a HPR-like variant that preserves HPR-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated HPR proteins, and biologically active portions thereof, or derivatives, fragments, analogs or homologs thereof. Also provided are polypeptide fragments suitable for use as immunogens to raise anti-HPR antibodies. In one embodiment, native HPR proteins can be isolated from cells or tissue sources by an appropriate purification scheme using standard protein purification techniques. In another embodiment, HPR proteins are produced by recombinant DNA techniques. Alternative to recombinant expression, a HPR protein or polypeptide can be synthesized chemically using standard peptide synthesis techniques.

An “isolated” or “purified” protein or biologically active portion thereof is substantially free of cellular material or other contaminating proteins from the cell or tissue source from which the HPR protein is derived, or substantially free from chemical precursors or other chemicals when chemically synthesized. The language “substantially free of cellular material” includes preparations of HPR protein in which the protein is separated from cellular components of the cells from which it is isolated or recombinantly produced. In one embodiment, the language “substantially free of cellular material” includes preparations of HPR protein having less than about 30% (by dry weight) of non-HPR protein (also referred to herein as a “contaminating protein”), more preferably less than about 20% of non-HPR protein, still more preferably less than about 10% of non-HPR protein, and most preferably less than about 5% non-HPR protein. When the HPR protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of HPR protein in which the protein is separated from chemical precursors or other chemicals that are involved in the synthesis of the protein. In one embodiment, the language “substantially free of chemical precursors or other chemicals” includes preparations of HPR protein having less than about 30% (by dry weight) of chemical precursors or non-HPR chemicals, more preferably less than about 20% chemical precursors or non-HPR chemicals, still more preferably less than about 10% chemical precursors or non-HPR chemicals, and most preferably less than about 5% chemical precursors or non-HPR chemicals.

Biologically active portions of a HPR protein include peptides comprising amino acid sequences sufficiently homologous to or derived from the amino acid sequence of the HPR protein, e.g., the amino acid sequence shown in SEQ ID NO:2 that include fewer amino acids than the full length HPR proteins, and exhibit at least one activity of a HPR protein, e.g. substrate binding. Typically, biologically active portions comprise a domain or motif with at least one activity of the HPR protein. A biologically active portion of a HPR protein can be a polypeptide which is, for example, 10, 25, 50, 100 or more amino acids in length.

A biologically active portion of a HPR protein of the present invention may contain at least one of the above-identified domains conserved between the HPR proteins, e.g. Moreover, other biologically active portions, in which other regions of the protein are deleted, can be prepared by recombinant techniques and evaluated for one or more of the functional activities of a native HPR protein.

A biologically active portion or a HPR protein can be the N-terminal domain of the HPR polypeptide. Alternatively, a biologically active portion or a HPR protein can be the C-terminal domain of the HPR polypeptide. Preferably, the biologically active portion comprises at least 75 amino acids of the C-terminal domain. More preferably, the biologically active portion comprises at least 25 amino acids of the C-terminal domain. Most preferably, the biologically active portion comprises at least 10 amino acids of the C-terminal.

In an embodiment, the HPR protein has an amino acid sequence shown in SEQ ID NO:2. In other embodiments, the HPR protein is substantially homologous to SEQ ID NO:2 and retains the functional activity of the protein of SEQ ID NO:2, yet differs in amino acid sequence due to natural allelic variation or mutagenesis, as described in detail below. Accordingly, in another embodiment, the HPR protein is a protein that comprises an amino acid sequence at least about 45% homologous to the amino acid sequence of S SEQ ID NO:2 and retains the functional activity of the HPR proteins of SEQ ID NO:2.

Determining Homology Between Two or More Sequence

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO:1 .

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues. Chimeric and fusion proteins

The invention also provides HPR chimeric or fusion proteins. As used herein, a HPR “chimeric protein” or “fusion protein” comprises a HPR polypeptide operatively linked to a non-HPR polypeptide. An “HPR polypeptide” refers to a polypeptide having an amino acid sequence corresponding to HPR, whereas a “non-HPR polypeptide” refers to a polypeptide having an amino acid sequence corresponding to a protein that is not substantially homologous to the HPR protein, e.g., a protein that is different from the HPR protein and that is derived from the same or a different organism. Within a HPR fusion protein the HPR polypeptide can correspond to all or a portion of a HPR protein. In one embodiment, a HPR fusion protein comprises at least one biologically active portion of a HPR protein. In another embodiment, a HPR fusion protein comprises at least two biologically active portions of a HPR protein. Within the fusion protein, the term “operatively linked” is intended to indicate that the HPR polypeptide and the non-HPR polypeptide are fused in-frame to each other. The non-HPR polypeptide can be fused to the N-terminus or C-terminus of the HPR polypeptide.

A HPR chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A HPR-encoding nucleic acid can be cloned into such an expression vector such that the fusion moiety is linked in-frame to the HPR protein.

HPR Antibodies

HPR polypeptides, including chimeric polypeptides, or derivatives, fragments, analogs or homologs thereof, may be utilized as immunogens to generate antibodies that immunospecifically-bind these peptide components. Such antibodies include, e.g., polyclonal, monoclonal, chimeric, single chain, Fab fragments and a Fab expression library. In a specific embodiment, fragments of the HPR polypeptides are used as immunogens for antibody production. Various procedures known within the art may be used for the production of polyclonal or monoclonal antibodies to a HPR polypeptides, or derivative, fragment, analog or homolog thereof.

For the production of polyclonal antibodies, various host animals may be immunized by injection with the native peptide, or a synthetic variant thereof, or a derivative of the foregoing. Various adjuvants may be used to increase the immunological response and include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.) and human adjuvants such as Bacille Calmette-Guerin and Corynebacterium parvuni.

For preparation of monoclonal antibodies directed towards a HPR polypeptides, or derivatives, fragments, analogs or homologs thereof, any technique that provides for the production of antibody molecules by continuous cell line culture may be utilized. Such techniques include, but are not limited to, the hybridoma technique (see, Kohler and Milstein, 1975. Nature 256: 495-497); the trioma technique; the human B-cell hybridoma technique (see, Kozbor, et al., 1983. Immunol Today 4: 72) and the EBV hybridoma technique to produce human monoclonal antibodies (see, Cole, et al., 1985. In: Monoclonal Antibodies and Cancer Therapy, Alan R. Liss, Inc., pp. 77-96). Human monoclonal antibodies may be utilized in the practice of the present invention and may be produced by the use of human hybridomas (see, Cote, et al., 1983. Proc Natl Acad Sci USA 80: 2026-2030) or by transforming human B-cells with Epstein Barr Virus in vitro (see, Cole, et al., 1985. In Monoclonal Antibodies and Cancer Therapy (Alan R. Liss, Inc., pp. 77-96).

According to the invention, techniques can be adapted for the production of single-chain antibodies specific to a HPR polypeptides (see, e.g., U.S. Pat. No. 4,946,778). In addition, methodologies can be adapted for the construction of Fab expression libraries (see, e.g., Huse, et a., 1989. Science 246: 1275-1281) to allow rapid and effective identification of monoclonal Fab fragments with the desired specificity for a HPR polypeptides or derivatives, fragments, analogs or homologs thereof Antibody fragments that contain the idiotypes to a HPR polypeptides may be produced by techniques known in the art including, e.g., (i) an F(ab′)₂ fragment produced by pepsin digestion of an antibody molecule; (ii) an Fab fragment generated by reducing the disulfide bridges of an F(ab′)₂ fragment; (iii) an Fab fragment generated by the treatment of the antibody molecule with papain and a reducing agent and (iv) Fv fragments.

In one embodiment, methodologies for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a HPR polypeptides is facilitated by generation of hybridomas that bind to the fragment of a HPR polypeptides possessing such a domain. Antibodies that are specific for a domain within a HPR polypeptides, or derivative, fragments, analogs or homologs thereof, are also provided herein. The anti-HPR polypeptide antibodies may be used in methods known within the art relating to the localization and/or quantitation of a HPR polypeptide(e.g., for use in measuring levels of the peptide within appropriate physiological samples, for use in diagnostic methods, for use in imaging the peptide, and the like).

HPR Recombinant Expression Vectors and Host Cells

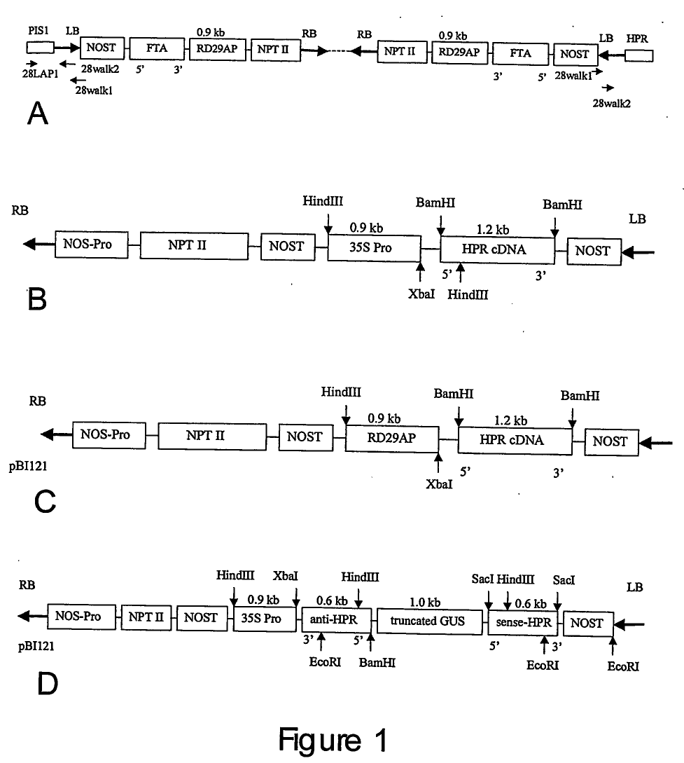

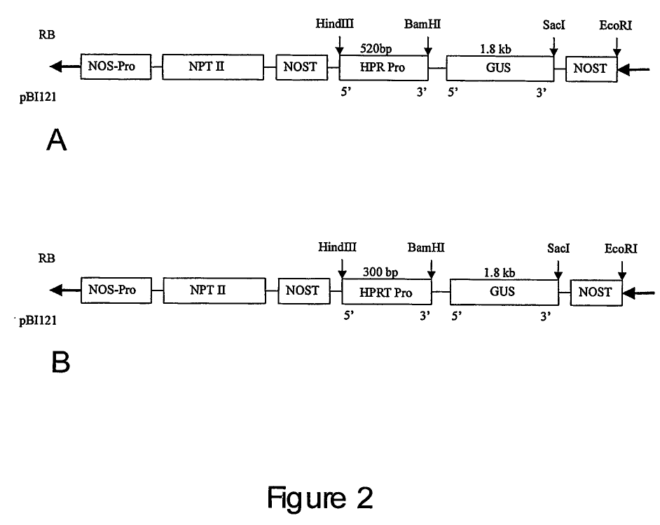

Another aspect of the invention pertains to vectors, preferably expression vectors, containing a nucleic acid encoding a HPR protein, or derivatives, fragments, analogs or homologs thereof. As used herein, the term “vector” refers to a nucleic acid molecule capable of transporting another nucleic acid to which it has been linked. Exemplary expression vector constructs include for example the constructs illustrated in FIG. 1 and FIG. 2. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication). Other vectors are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors or plant transformation vectors, binary or otherwise, which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the. vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). Examples of suitable promoters include for example constitutive promoters, ABA inducible promoters, tissue specific promters or guard cell specific promoters. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., HPR proteins, mutant forms of HPR proteins, fusion proteins, etc.).

The recombinant expression vectors of the invention can be designed for expression of HPR proteins in prokaryotic or eukaryotic cells. For example, HPR proteins can be expressed in bacterial cells such as Escherichia coli, insect cells (using baculovirus expression vectors) yeast cells, plant cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterolinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et.al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the HPR expression vector is a yeast expression vector. Examples of vectors for expression in yeast Saccharomyces cerivisae include pYepSec1 (Baldari, et al., 1987. EMBO J. 6: 229-234), pMFa (uijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, HPR can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In yet another embodiment, a nucleic acid of the invention is expressed in plants cells using a plant expression vector. Examples of plant expression vectors systems include tumor inducing (Ti) plasmid or portion thereof found in Agrobacterium, cauliflower mosaic virus (CAMV) DNA and vectors such as pBI121 .

For expression of HPR in plants, the recombinant expression cassette will contain in addition to the HPR nucleic acids, a plant promoter region, a transcription initiation site (if the coding sequence to transcribed lacks one), and a transcription termination/polyadenylation sequence. The termination/polyadenylation region may be obtained from the same gene as the promoter sequence or may be obtained from different genes. Unique restriction enzyme sites at the 5′ and 3′ ends of the cassette are typically included to allow for easy insertion into a pre-existing vector.

Examples of suitable promotors include promoters from plant viruses such as the 35S promoter from cauliflower mosaic virus (CaMV). Odell, et al., Nature, 313: 810-812 (1985). and promoters from genes such as rice actin (McElroy, et al., Plant Cell, 163-171 (1990)); ubiquitin (Christensen, et al., Plant Mol. Biol., 12: 619-632 (1992); and Christensen, et al., Plant Mol. Biol., 18: 675-689 (1992)); pEMU (Last, et al., Theor. Appl. Genet., 81: 581-588 (1991)); MAS (Velten, et al., EMBO J., 3: 2723-2730 (1984)); maize H3 histone (Lepetit, et al., Mol. Gen. Genet., 231: 276-285 (1992); and Atanassvoa, et al., Plant Journal, 2(3): 291-300 (1992)), the 5′- or 3′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, ALS promoter, (WO 96/30530), a synthetic promoter, such as, Rsyn7, SCP and UCP promoters, ribulose-1,3-diphosphate carboxylase, fruit-specific promoters, heat shock promoters, seed-specific promoters and other transcription initiation regions from various plant genes, for example, include the various opine initiation regions, such as for example, octopine, mannopine, and nopaline.

Additional regulatory elements that may be connected to a HPR encoding nucleic acid sequence for expression in plant cells include terminators, polyadenylation sequences, and nucleic acid sequences encoding signal peptides that permit localization within a plant cell or secretion of the protein from the cell. Such regulatory elements and methods for adding or exchanging these elements with the regulatory elements HPR gene are known, and include, but are not limited to, 3′ termination and/or polyadenylation regions such as those of the Agrobacterium tumefaciens nopaline synthase (nos) gene (Bevan, et al., Nucl. Acids Res., 12: 369-385 (1983)); the potato proteinase inhibitor II (PINII) gene (Keil, et al., Nucl. Acids Res., 14: 5641-5650 (1986) and hereby incorporated by reference); and An, et al., Plant Cell, 1: 115-122 (1989)); and the CaMV 19S gene (Mogen, et al., Plant Cell, 2: 1261-1272 (1990)).

Plant signal sequences, including, but not limited to, signal-peptide encoding DNA/RNA sequences which target proteins to the extracellular matrix of the plant cell (Dratewka-Kos, et al., J. Biol. Chem., 264: 4896-4900 (1989)) and the Nicotiana plumbaginifolia extension gene (DeLoose, et al., Gene, 99: 95-100 (1991)), or signal peptides which target proteins to the vacuole like the sweet potato sporamin gene (Matsuka, et al., Proc. Nat'l Acad. Sci. (ISA), 88: 834 (1991)) and the barley lectin gene (Wilkins, et al., Plant Cell, 2: 301-313 (1990)), or signals which cause proteins to be secreted such as that of PRIb (Lind, et al., Plant Mol. Biol., 18: 47-53 (1992)), or those which target proteins to the plastids such as that of rapeseed enoyl-ACP reductase (Verwaert, et al., Plant Mol. Biol., 26: 189-202 (1994)) are useful in the invention.

In another embodiment, the recombinant expression vector is capable of directing expression of the nucleic acid preferentially in a particular cell type (e.g., tissue-specific regulatory elements are used to express the nucleic acid). Tissue-specific regulatory elements are known in the art. Especially useful in connection with the nucleic acids of the present invention are expression systems which are operable in plants. These include systems which are under control of a tissue-specific promoter, as well as those which involve promoters that are operable in all plant tissues.

Organ-specific promoters are also well known. For example, the patatin class I promoter is transcriptionally activated only in the potato tuber and can be used to target gene expression in the tuber (Bevan, M., 1986, Nucleic Acids Research 14:4625-4636). Another potato-specific promoter is the granule-bound starch synthase (GBSS) promoter (Visser, R. G. R, et al., 1991, Plant Molecular Biology 17:691-699).

Other organ-specific promoters appropriate for a desired target organ can be isolated using known procedures. These control sequences are generally associated with genes uniquely expressed in the desired organ. In a typical higher plant, each organ has thousands of mRNAs that are absent from other organ systems (reviewed in Goldberg, P., 1986, Trans. R. Soc. London B314:343).

For in situ production of the antisense mRNA of HPR, those regions of the HPR gene which are transcribed into HPR mRNA, including the untranslated regions thereof, are inserted into the expression vector under control of the promoter system in a reverse orientation. The resulting transcribed mRNA is then complementary to that normally produced by the plant.

The resulting expression system or cassette is ligated into or otherwise constructed to be included in a recombinant vector which is appropriate for plant transformation. The vector may also contain a selectable marker gene by which transformed plant cells can be identified in culture. Usually, the marker gene will encode antibiotic resistance. These markers include resistance to G418, hygromycin, bleomycin, kanamycin, and gentamicin. After transforming the plant cells, those cells having the vector will be identified by their ability to grow on a medium containing the particular antibiotic. Replication sequences, of bacterial or viral origin, are generally also included to allow the vector to be cloned in a bacterial or phage host, preferably a broad host range prokaryotic origin of replication is included. A selectable marker for bacteria should also be included to allow selection of bacterial cells bearing the desired construct. Suitable prokaryotic selectable markers also include resistance to antibiotics such as kanamycin or tetracycline.

Other DNA sequences encoding additional functions may also be present in the vector, as is known in the art. For instance, in the case of Agrobacterium transformations, T-DNA sequences will also be included for subsequent transfer to plant chromosomes.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell.

A host cell of the invention, such as a prokaryotic or eukaryotic host cell in culture, can be used to produce (i.e., express) a polypeptide of the invention encoded in a an open reading frame of a polynucleotide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

A number of types of cells may act as suitable host cells for expression of a polypeptide encoded by an open reading frame in a polynucleotide of the invention. Plant host cells include, for example, plant cells that could function as suitable hosts for the expression of a polynucleotide of the invention include epidermal cells, mesophyll and other ground tissues, and vascular tissues in leaves, stems, floral organs, and roots from a variety of plant species, such as Arabidopsis thaliana, Nicotiana tabacum, Brassica napus, Zea mays, Oryza sativa, Gossypium hirsutum and Glycine max.

Alternatively, it may be possible to produce a polypeptide in lower eukaryotes such as yeast or in prokaryotes such as bacteria. Potentially suitable yeast strains include Saccharomiyces cerevisiae, Schizosaccharomyces pombe, Kluyveromyces strains, Candida, or any yeast strain capable of expressing heterologous proteins. Potentially suitable bacterial strains include Eschlerichia coli, Bacillus subtilis, Salmonella typhiniururn, or any bacterial strain capable of expressing heterologous polypeptides. If the polypeptide is made in yeast or bacteria, it may be necessary to modify the polypeptide produced therein, for example by phosphorylation or glycosylation of the appropriate sites, in order to obtain a functional polypeptide, if the polypeptide is of sufficient length and conformation to have activity. Such covalent attachments may be accomplished using known chemical or enzymatic methods.

A polypeptide may be prepared by culturing transformed host cells under culture conditions suitable to express the recombinant protein. The resulting expressed polypeptide or protein may then be purified from such culture (e.g., from culture medium or cell extracts) using known purification processes, such as gel filtration and ion exchange chromatography. The purification of the polypeptide or protein may also include an affinity column containing agents which will bind to the protein; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography.

Alternatively, a polypeptide or protein may also be expressed in a form which will facilitate purification. For example, it may be expressed as a fusion protein containing a six-residue histidine tag. The histidine-tagged protein will then bind to a Ni-affinity column. After elution of all other proteins, the histidine-tagged protein can be eluted to achieve rapid and efficient purification. One or more reverse-phase high performance liquid chromatography (RP-HPLC) steps employing hydrophobic RP-HPLC media, e.g., silica gel having pendant methyl or other aliphatic groups, can be employed to further purify a polypeptide. Some or all of the foregoing purification steps, in various combinations, can also be employed to provide a substantially homogeneous isolated recombinant polypeptide. The protein or polypeptide thus purified is substantially free of other plant proteins or polypeptides and is defined in accordance with the present invention as “isolated.”

Transformed Plants Cells and Transgenic Plants

The invention includes protoplast, plants cells, plant tissue and plants (e.g., monocots and dicots transformed with a HPR promoter-gene construct, a vector (e.g., expression vector) containing a HPR promoter-gene construct, a HPR nucleic acid (i.e, sense or antisense), a vector containing a HPR nucleic acid (i.e, sense or antisense)or an expression vector containing a HPR nucleic acid (i.e, sense or antisense). As used herein, “plant” is meant to include not only a whole plant but also a portion thereof (ie., cells, and tissues, including for example, leaves, stems, shoots, roots, flowers, fruits and seeds).

The plant can be any plant type including, for example, species from the genera Cucurbita, Rosa, Vitis, Juglans, Fragaria, Lotus, Medicago, Onobrychis, Trifolium, Trigonella, Vigiza, Citrus, Linumn, Geranium, Mainihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Peynisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Gossypium, Picea, Caco, and Populus.

In some aspects of the invention, the transformed plant is resistant to biotic and abiotic stresses, e.g., chilling stress, heat stress, salt stress, water stress (e.g., drought), photo-oxidative stress, disease, grazing pests and wound healing. Additionally, the invention also includes a transgenic plant that is resistant to pathogens such as for example fungi, bacteria, nematodes, viruses and parasitic weeds. Alternatively, the transgenic plant is resistant to herbicides or has altered senescence. The transgenic plant has an increase in yield, productivity, biomass or ABA sensitivity. By resistant is meant the plant grows under stress conditions (e.g., high salt, decreased water, low temperatures) or under conditions that normally inhibit, to some degree, the growth of an untransformed plant. Methodologies to determine plant growth or response to stress include for example, height measurements, weight measurements, leaf area, ability to flower, water use, transpiration rates and yield.

The invention also includes cells, tissues, including for example, leaves, stems, shoots, roots, flowers, fruits and seeds and the progeny derived from the tranformed plant.

Numerous methods for introducing foreign genes into plants are known and can be used to insert a gene into a plant host, including biological and physical plant transformation protocols. See, for example, Miki et al., (1993) “Procedure for Introducing Foreign DNA into Plants”, In: Methods in Plant Molecular Biology and Biotechnology, Glick and Thompson, eds., CRC Press, Inc., Boca Raton, pages 67-88 and Andrew Bent in, Clough S J and Bent A F, 1998. Floral dipping: a simplified method for Agrobacterium-mediated transformation of Arabidopsis thaliana. The methods chosen vary with the host plant, and include chemical transfection methods such as calcium phosphate, polyethylene glycol (PEG) transformation, microorganism-mediated gene transfer such as Agrobacterium (Horsch, et al., Science, 227: 1229-31 (1985)), electroporation, protoplast transformation, micro-injection, flower dipping and biolistic bombardment.

Agrobacterium-Mediated Transformation

The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectfully, carry genes responsible for genetic transformation of plants. See, for example, Kado, Crit. Rev. Plant Sci., 10: 1-32 (1991). Descriptions of the Agrobacterium vector systems and methods for Agrobacterium-mediated gene transfer are provided in Gruber et al., supra; and Moloney, et al, Plant Cell Reports, 8: 238-242 (1989).

Transgenic Arabidopsis plants can be produced easily by the method of dipping flowering plants into an Agrobacterium culture, based on the method of Andrew Bent in, Clough S J and Bent A F, 1998. Floral dipping: a simplified method for Agrobacterium-mediated transformation of Arabidopsis thaliana. Wild type plants are grown until the: plant has both developing flowers and open flowers. The plant are inverted for 1 minute into a solution of Agrobacterium culture carrying the appropriate gene construct. Plants are then left horizontal in a tray and kept covered for two days to maintain humidity and then righted and bagged to continue growth and seed development. Mature seed is bulk harvested.

Direct Gene Transfer

A generally applicable method of plant transformation is microprojectile-mediated transformation, where DNA is carried on the surface of microprojectiles measuring about 1 to 4 mu.m. The expression vector is introduced into plant tissues with a biolistic device that accelerates the microprojectiles to speeds of 300 to 600 m/s which is sufficient to penetrate the plant cell walls and membranes. (Sanford, et al., Part. Sci. Technol., 5: 27-37 (1987); Sanford, Trends Biotech, 6: 299-302 (1988); Sanford, Physiol. Plant, 79: 206-209 (1990); Klein, et al., Biotechnology, 10: 286-291 (1992)).

Another method for physical delivery of DNA to plants is sonication of target cells as described in Zang, et al., BioTechnology, 9: 996-996 (1991). An alternative transformation method utilizing sonication is described in U.S. Pat. No. 5,693,512 as described by Finer, J. J. and Trick, H. N. Alternatively, liposome or spheroplast fusions have been used to introduce expression vectors into plants. See, for example, Deshayes, et al., EMBO J., 4: 2731-2737 (1985); and Christou, et al., Proc. Nat'l. Acad. Sci. (USA), 84: 3962-3966 (1987). Direct uptake of DNA into protoplasts using CaCl.sub.2 precipitation, polyvinyl alcohol or poly-L-ornithine have also been reported. See, for example, Hain, et al., Mol. Gen. Genet., 199: 161 (1985); and Draper, et al., Plant Cell Physiol., 23: 451-458 (1982).

Electroporation of protoplasts and whole cells and tissues has also been described. See, for example, Donn, et al., (1990) In: Abstracts of the VIIth Int;l. Congress on Plant Cell and Tissue Culture IAPTC, A2-38, page 53; D'Halluin et al., Plant Cell, 4: 1495-1505 (1992); and Spencer et al., Plant Mol. Biol., 24: 51-61 (1994).

Alternatively one can use a non-particle biolistic bombardment transformation method. An example of non-particle biolistic transformation is given in U.S. Patent Application 20010026941. This method has been used to produce transgenic Glycine max and Zea maize plants. Viable plants are propagated and homozygous lines are generated.

Particle Wounding/Agrobacterium Delivery

Another useful basic transformation protocol involves a combination of wounding by particle bombardment, followed by use of Agrobacterium for DNA delivery, as described by Bidney, et al., Plant Mol. Biol., 18: 301-31 (1992). Useful plasmids for plant transformation include Bin 19. See Bevan, Nucleic Acids Research, 12: 8711-8721 (1984), and hereby incorporated by reference.

In general, the intact meristem transformation method involves imbibing seed for 24 hours in the dark, removing the cotyledons and root radical, followed by culturing of the meristem explants. Twenty-four hours later, the primary leaves are removed to expose the apical meristem. The explants are placed apical dome side up and bombarded, e.g., twice with particles, followed by co-cultivation with Agrobacterium. To start the co-cultivation for intact meristems, Agrobacterium is placed on the meristem. After about a 3-day co-cultivation period the meristems are transferred to culture medium with cefotaxime plus kanamycin for the NPTII selection.

The split meristem method involves imbibing seed, breaking of the cotyledons to produce a clean fracture at the plane of the embryonic axis, excising the root tip and then bisecting the explants longitudinally between the primordial leaves. The two halves are placed cut surface up on the medium then bombarded twice with particles, followed by co-cultivation with Agrobacterium. For split meristems, after bombardment, the meristems are placed in an Agrobacterium suspension for 30 minutes. They are then removed from the suspension onto solid culture medium for three day co-cultivation. After this period, the meristems are transferred to fresh medium with cefotaxime plus kanamycin for selection.

Transfer by Plant Breeding

Alternatively, once a single transformed plant has been obtained by the foregoing recombinant DNA method, conventional plant breeding methods can be used to transfer the gene and associated regulatory sequences via crossing and backcrossing. Such intermediate methods will comprise the further steps of: (1) sexually crossing the transgenic plant with a plant from a second taxon; (2) recovering reproductive material from the progeny of the cross; and (3) growing transgenic plants from the reproductive material. Where desirable or necessary, the agronomic characteristics of the second taxon can be substantially preserved by expanding this method to include the further steps of repetitively: (1) backcrossing the transgenic progeny with non-transgenic plants from the second taxon; and (2) selecting for expression of an associated marker gene among the progeny of the backcross, until the desired percentage of the characteristics of the second taxon are present in the progeny along with the gene or genes imparting marker gene trait.

By the term “taxon” herein is meant a unit of botanical classification. It thus includes, genus, species, cultivars, varieties, variants and other minor taxonomic groups which lack a consistent nomenclature.

Regeneration of Transformants

The development or regeneration of plants from either single plant protoplasts or various explants is well known in the art (Weissbach and Weissbach, 1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a polypeptide of interest introduced by Agrobacterium from leaf explants can be achieved by methods well known in the art such as described (Horsch et al., 1985). In this procedure, transformants are cultured in the presence of a selection agent and in a medium that induces the regeneration of shoots in the plant strain being transformed as described (Fraley et al., 1983). In particular, U.S. Pat. No. 5,349,124 (specification incorporated herein by reference) details the creation of genetically transformed lettuce cells and plants resulting therefrom which express hybrid crystal proteins conferring insecticidal activity against Lepidopteran larvae to such plants.

This procedure typically produces shoots within two to four months and those shoots are then transferred to an appropriate root-inducing medium containing the selective agent and an antibiotic to prevent bacterial growth. Shoots that rooted in the presence of the selective agent to form plantlets are then transplanted to soil or other media to allow the production of roots. These procedures vary depending upon the particular plant strain employed, such variations being well known in the art.

Preferably, the regenerated plants are self-pollinated to provide homozygous transgenic plants, or pollen obtained from the regenerated plants is crossed to seed-grown plants of agronomically important, preferably inbred lines. Conversely, pollen from plants of those important lines is used to pollinate regenerated plants. A transgenic plant of the present invention containing a desired polypeptide is cultivated using methods well known to one skilled in the art.

A preferred transgenic plant is an independent segregant and can transmit the HPR gene and its activity to its progeny. A more preferred transgenic plant is homozygous for the gene, and transmits that gene to all of its offspring on sexual mating. Seed from a transgenic plant may be grown in the field or greenhouse, and resulting sexually mature transgenic plants are self-pollinated to generate true breeding plants. The progeny from these plants become true breeding lines that are evaluated for increased expression of the HPR transgene.

Method of Producing Transgenic Plants

Also included in the invention are methods of producing a transgenic plant.

A transgenic plant is produced by introducing into one or more plant cells a HPR promoter-gene construct according to the invention (as described above) to generate a transgenic cell and regenerating a transgenic plant from the transgenic cell. The transgenic plant has an altered level of expression of a protein of interest. For example, expression of the protein level is at an increased level compared to a normal control level. Alternatively, the transgenic plant expresses a protein of interest at a decreased level compared a normal control level. By normal control level is meant a level of gene/protein expression detected in a normal, untransformed plant (i.e., wild-type).

For example, when a plant cell is transformed with a HPR-gene construct containing a non-translatable mRNA molecule of a gene encoding a protein of interest, the resulting transgenic plant has a decreased level of expression compared to a wild-type plant. Similarly, when a plant cell is transformed with a HPR-gene construct containing a nucleic acid encoding for a gene of interest, the resulting transgenic plant has an increase level of expression compared to a wild-type plant.

Expression of the protein of interest is detected and measured at the nucleic acid level using techniques well known to one of ordinary skill in the art and include amplification-based detection methods such as reverse-transcription based polymerase chain reaction. Expression of he protein of interest is also determined at the protein level, i.e., by measuring the levels of polypeptides encoded by the gene products. Such methods are well known in the art and include, e.g., immunoassays based on antibodies to proteins encoded by the genes.

Transgenic plants are also produced by introducing into one or more plant cells a compound that alters hydroxypyruvate reductase expression or activity in the plant to generate a transgenic plant cell and regenerating a transgenic plant from the transgenic cell. In some aspects the compound increases alters hydroxypyruvate reductase expression or activity. Alternatively, the compound decrease alters hydroxypyruvate reductase expression or activity. The compound can be, e.g., (i) a hydroxypyruvate reductase polypeptide; (ii) a nucleic acid encoding a hydroxypyruvate reductase polypeptide; (iii) a nucleic acid that increases expression of a nucleic acid that encodes a hydroxypyruvate reductase polypeptide ; (iv) a nucleic acid that decreases the expression of a nucleic acid that encodes a hydroxypyruvate reductase polypeptide; (v) a hydroxypyruvate reductase antisense nucleic acid and derivatives, fragments, analogs and homologs thereof A nucleic acid that increases expression of a nucleic acid that encodes a hydroxypyruvate reductase polypeptide includes, e.g., promoters, enhancers. The nucleic acid can be either endogenous or exogenous. Preferably, the compound is a hydroxypyruvate reductase polypeptide or a nucleic acid encoding a hydroxypyruvate reductase polypeptide. For example the compound comprises the nucleic acid sequence of SEQ ID NO:1 or a fragment thereof. Alternatively, the compound is a hydroxypyruvate reductase antisense nucleic acid. For example the compound comprises the nucleic acid sequence of SEQ ID NO:3.

In various aspects the transgenic plant produced by the methods of the invention has an altered phenotype as compared to a wild type plant (i.e., untransformed). By altered phenotype is meant that the plant has a one or more characteristic that is different from the wild type plant. For example, the transgenic plant has an increased resistence to stress. Increased stress resistance is meant that the transgenic plant can grow under stress conditions (e.g., high salt, decreased water, low temperatures, high temperatures) or under conditions that normally inhibit the growth of an untransformed Stresses include, for example, chilling stress, heat stress, heat shock, salt stress, water stress (i.e, drought), photo-oxidative stress, nutritional stress, disease, grazing pests, wound healing, pathogens such as for example fungi, bacteria, nematodes, viruses or parasitic weed and herbicides. Methodologies to determine plant growth or response to stress include for example, height measurements, weight or biomass measurements, leaf area or number, ability to flower, water use, transpiration rates and yield. Alternatively, the transformed plant has an increased (i.e., enhanced) ABA sensitivity. The enhanced ABA sensitivity is at the seedling growth stage. Alternatively, the enhanced ABA sensitivity is at the mature plant stage. Additional altered phenotypes include for example, enhanced vegetative growth (e.g., increased leaf number, thickness and overall biomass), delayed reproductive growth (e.g., flowering later); enhanced seedling vigor (e.g., increased root biomass and length), enhanced lateral root formation and therefore soil penetration more extensive vascular system resulting in an enhanced transport system.

The plant cell from a dicot or a monocot plant. The plant is any plant type including, for example, species from the genera Cucurbita, Rosa, Vitis, Juglans, Fragaria, Lotus, Medicago, Otiobrychis, Tnifolium, Trigonella, Vigna, Citrus, Linum, Geranium, Manihot, Daucus, Arabidopsis, Brassica, Raphaizus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Paniieum, Pennisetum, Raizunculus, Senecio, Salpiglossis, Cucumis, Browaalia, Glycine, Pisum, Phaseolus, Lolium, Oryza, Zea, Avena, Hordeum, Secale, Triticum, Sorghum, Gossypium, Picea, Caco, and Populus.

Screening Methods

The isolated nucleic acid molecules of the invention can be used to express HPR protein (e.g., via a recombinant expression vector in a host cell), to detect HPR mRNA (e.g., in a biological sample) or a genetic lesion in a HPR gene, and to modulate HPR activity, as described further, below. In addition, the HPR proteins can be used to screen compounds that modulate the HPR protein activity or expression. In addition, the anti-HPR antibodies of the invention can be used to detect and isolate HPR proteins and modulate HPR activity.

The invention provides a method (also referred to herein as a “screening assay”) for identifying modulators, i.e., candidate or test compounds or agents (e.g., peptides, peptidomimetics, small molecules or other drugs) that bind to HPR proteins or have a stimulatory or inhibitory effect on, e.g., HPR protein expression or HPR protein activity. The invention also includes compounds identified in the screening assays described herein.

In one embodiment, the invention provides assays for screening candidate or test compounds which bind to a HPR protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Designi 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. US.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a HPR protein, or a biologically-active portion thereof, is contacted with a test compound and the ability of the test compound to bind to a HPR protein determined. The cell, for example, can be of mammalian origin, plant cell or a yeast cell. Determining the ability of the test compound to bind to the HPR protein can be accomplished, for example, by coupling the test compound with a radioisotope or enzymatic label such that binding of the test compound to the HPR protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a HPR protein, or a biologically-active portion thereof, with a known compound which binds HPR to form an assay mixture, contacting the assay mixture with a test compound, and determining the ability of the test compound to interact with a HPR protein, wherein determining the ability of the test compound to interact with a HPR protein comprises determining the ability of the test compound to preferentially bind to HPR protein or a biologically-active portion thereof as compared to the known compound.

In another embodiment, an assay is a cell-based assay comprising contacting a cell expressing a HPR protein, or a biologically-active portion thereof, with a test compound and determining the ability of the test compound to modulate (e.g. stimulate or inhibit) the activity of the HPR protein or biologically-active portion thereof. Determining the ability of the test compound to modulate the activity of HPR or a biologically-active portion thereof can be accomplished, for example, by determining the ability of the HPR protein to bind to or interact with a HPR target molecule. As used herein, a “target molecule” is a molecule with which a HPR protein binds or interacts in nature, for example, a molecule on the surface of a cell which expresses a HPR interacting protein, a molecule on the surface of a second cell, a molecule in the extracellular milieu, a molecule associated with the internal surface of a cell membrane or a cytoplasmic molecule. A HPR target molecule can be a non-HPR molecule or a HPR protein or polypeptide of the invention In one embodiment, a HPR target molecule is a component of a signal transduction pathway that facilitates transduction of an extracellular signal (e.g. a signal generated by binding of a compound to a membrane-bound molecule) through the cell membrane and into the cell. The target, for example, can be a second intercellular protein that has catalytic activity or a protein that facilitates the association of downstream signaling molecules with HPR.

Determining the ability of the HPR protein to bind to or interact with a HPR target molecule can be accomplished by one of the methods described above for determining direct binding. In one embodiment, determining the ability of the HPR protein to bind to or interact with a HPR target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a HPR-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a HPR protein or biologically-active portion thereof with a test compound and determining the ability of the test compound to bind to the HPR protein or biologically-active portion thereof. Binding of the test compound to the HPR protein can be determined either directly or indirectly as described above. In one such embodiment, the assay comprises contacting the HPR protein or biologically-active portion thereof with a known compound which binds HPR to form an assay mixture, contacting the assay mixture with a test compound, and determining the ability of the test compound to interact with a HPR protein, wherein determining the ability of the test compound to interact with a HPR protein comprises determining the ability of the test compound to preferentially bind to HPR or biologically-active portion thereof as compared to the known compound.

In still another embodiment, an assay is a cell-free assay comprising contacting HPR protein or biologically-active portion thereof with a test compound and determining the ability of the test compound to modulate (e.g. stimulate or inhibit) the activity of the HPR protein or biologically-active portion thereof. Determining the ability of the test compound to modulate the activity of HPR can be accomplished, for example, by determining the ability of the HPR protein to bind to a HPR target molecule by one of the methods described above for determining direct binding. In an alternative embodiment, determining the ability of the test compound to modulate the activity of HPR protein can be accomplished by determining the ability of the HPR protein further modulate a HPR target molecule. For example, the catalytic/enzymatic activity of the target molecule on an appropriate substrate can be determined as described above.

In yet another embodiment, the cell-free assay comprises contacting the HPR protein or biologically-active portion thereof with a known compound which binds HPR protein to form an assay mixture, contacting the assay mixture with a test compound, and determining the ability of the test compound to interact with a HPR protein, wherein determining the ability of the test compound to interact with a HPR protein comprises determining the ability of the HPR protein to preferentially bind to or modulate the activity of a HPR target molecule.

The cell-free assays of the invention are amenable to use of both the soluble form or the membrane-bound form of HPR protein. In the case of cell-free assays comprising the membrane-bound form of HPR protein, it may be desirable to utilize a solubilizing agent such that the membrane-bound form of HPR protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either HPR protein or its target molecule to facilitate separation of complexed from uncomplexed forms of one or both of the proteins, as well as to accommodate automation of the assay. Binding of a test compound to HPR protein, or interaction of HPR protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-HPR fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or HPR protein, and the mixture is incubated under conditions conducive to complex formation (e.g., at physiological conditions for salt and p”). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of HPR protein binding or activity determined using standard techniques.

Other techniques for immobilizing proteins on matrices can also be used in the screening assays of the invention. For example, either the HPR protein or its target molecule can be immobilized utilizing conjugation of biotin and streptavidin. Biotinylated HPR protein or target molecules can be prepared from biotin-NHS (N-hydroxy-succinimide) using techniques well-known within the art (e.g. biotinylation kit, Pierce Chemicals, Rockford, Ill.), and immobilized in the wells of streptavidin-coated 96 well plates (Pierce Chemical). Alternatively, antibodies reactive with HPR protein or target molecules, but which do not interfere with binding of the HPR protein to its target molecule, can be derivatized to the wells of the plate, and unbound target or HPR protein trapped in the wells by antibody conjugation. Methods for detecting such complexes, in addition to those described above for the GST-immobilized complexes, include immunodetection of complexes using antibodies reactive with the HPR protein or target molecule, as well as enzyme-linked assays that rely on detecting an enzymatic activity associated with the HPR protein or target molecule.

In another embodiment, modulators of HPR protein expression are identified in a method wherein a cell is contacted with a candidate compound and the expression of HPR mRNA or protein in the cell is determined. The level of expression of HPR mRNA or protein in the presence of the candidate compound is compared to the level of expression of HPR mRNA or protein in the absence of the candidate compound. The candidate compound can then be identified as a modulator of HPR mRNA or protein expression based upon this comparison. For example, when expression of HPR mRNA or protein is greater (i.e., statistically significantly greater) in the presence of the candidate compound than in its absence, the candidate compound is identified as a stimulator of HPR mRNA or protein expression. Alternatively, when expression of HPR mRNA or protein is less (statistically significantly less) in the presence of the candidate compound than in its absence, the candidate compound is identified as an inhibitor of HPR mRNA or protein expression. The level of HPR mRNA or protein expression in the cells can be determined by methods described herein for detecting HPR mRNA or protein.

In yet another aspect of the invention, the HPR proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with HPR (“HPR-binding proteins” or “HPR-bp”) and modulate HPR activity. Such HPR-binding proteins are also likely to be involved in the propagation of signals by the HPR proteins as, for example, upstream or downstream elements of the HPR pathway.

The two-hybrid system is based on the modular nature of most transcription factors, which consist of separable DNA-binding and activation domains. Briefly, the assay utilizes two different DNA constructs. In one construct, the gene that codes for HPR is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a HPR-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with HPR.

In yet another aspect of the invention are methods which utilize the transgenic plants of the invention to identify HPR-interacting components via genetic screening protocols. These components can be for example, regulatory elements which modify HPR-gene expression, interacting proteins which directly modify HPR activity or interacting proteins which modify components of the same signal transduction pathway and thereby exert an effect on the expression or activity of HPR. Briefly, genetic screening protocols are applied to the transgenic plants of the invention and in so doing identify related genes which are not identified using a wild type background for the screen. For example an activation tagged library (Weigel, et al., 2000. Plant Physiol. 122: 1003-1013), can be produced using the transgenic plants of the invention as the genetic background. Plants are then screened for altered phenotypes from that displayed by the parent plants. Alternative methods of generating libraries from the transgenic plants of the invention can be used, for example, chemical or irradiation induced mutations, insertional inactivation or insertional activation methods.

The invention further pertains to novel agents identified by the aforementioned screening assays and uses thereof.

### EXAMPLE 1

### Identification of line 28.1

Lines of Arabidopsis thaliana transgenic for the pRD29A-anti-FTA construct were screened for a drought tolerant phenotype. The phenotype displayed by this particular line was that of a drought susceptible plant. Additionally, the line was found to display ABA insensitive characteristics. The line 28.1 could represent an insertional mutant in a related gene of the ABA pathway and or the drought tolerance pathway.

### EXAMPLE 2

### Identification of HPR Gene

The site of the T-DNA insertion was identified using a PCR approach that amplified from a known site within the T-DNA to an annealed linker. A library of genomic DNA fragments was produced and screened as follows. Genomic DNA was isolated from Line 28.1 and digested by the restriction enzyme EcoRV. Two oligonucleotide adapters identified by SEQ ID NO:17 and SEQ ID NO:18 were mixed to a final concentration of 25 μM each. They were annealed to form a DNA linker by incubating at 95° C. for 1 min and cooling at room temperature. The linker was then ligated to the EcoRV digested genomic DNA.

A first PCR reaction utilized primers identified by SEQ ED NO:19 and SEQ ID NO:20. The sequence of SEQ ID NO:19 anneals at the ligated linker and SEQ ID NO:20 anneals to the T-DNA sequence inside of the left border of the pRD29A-anti-FTA construct in Line 28.1. Two major bands were produced, one of 1.1 kb and the second of 1.2 kb. The reaction mixture from the first PCR amplification was diluted 1/50 and re-amplified using nested primers identified by SEQ ID NO:21 and SEQ ID NO:22. Two major DNA fragments were produced, one was about 1 kb and the second was 860 bp. These final two DNA fragments were ligated into pBluescript T/A vector and sequenced.

Sequence analysis resulted in a match to a 860 bp fragment of the Arabidopsis chromosome 1, BAC clone T23K23, Genbank accession number AC012563. This region was identified and contained sequence from the cloned hydroxypyruvate reductase (HPR) coding sequence and upstream sequence predicted to be a promoter. It was concluded that the T-DNA had inserted into the promoter region of the HPR gene.

The insertion site was further characterized. Based on the genomic sequence data available from the Genbank database, the upstream region was determined to contain the gene phosphatidylinositol synthase (PIS1). Primers were produced, SEQ ID NO:23 and SEQ ID NO:26, and used to amplify portions of these genes and the intergenic region including the T-DNA insert. This primer set amplifies from within the PIS1 gene to a primer within the NPTII gene contained on the T-DNA construct of line 28.1. A larger than predicted fragment suggested that there was a tandem insertion of the T-DNA. Subsequent PCR reactions to amplify the upstream region indicated that these T-DNA insertions were in opposite orientations. Amplification of the upstream region was performed using primers identified by SEQ ID NO:23 and SEQ I) NO:20. Sequence analysis of the resulting PCR product contained the 3′ end of the PIS1 gene and the intergenic region and matched that present in the data base from Genbank Accession Number AC012563. The location of the T-DNA insertion was thereby determined.

### EXAMPLE 3

### Cloning of Arabidopsis HPR cDNA and Promoter and Vector Construction Using These Sequences

Using sequence data from the data base Accession Number AC012563, primers were designed to PCR amplify the HPR gene coding sequence. Total RNA was isolated from Arabidopsis leaf tissue using the QIAGEN RNeasy® Plant kit.

The primer pair identified by SEQ ID NO:6 and SEQ ID NO:7 were used to amplify the appropriate gene sequence. The DNA fragment was cloned into pBluescript T/A vector at the EcoRV site and sequenced. To facilitate the next cloning step the HPR gene was reamplified from the above plasmid using primer pairs SEQ ID NO:8 and SEQ ID NO:7. The resulting DNA fragment was digested with BamHI and cloned into pBI121, replacing the BamHI fragment encoding the GUS gene. The resulting vector is pBI121-HPR.

The primer pair SEQ ID NO:9 and SEQ ID NO:10 was used to PCR amplify the RD29A promoter sequence which replaced the 35S promoter of plasmid pBI121-HPR to generate plasmid pRD29A-HPR.

Construction of the vector pBI121-hp-HPR, a hair pin RNAi construct, was produced as follows. The cloning strategy involved truncating the GUS gene of pBI121 and flanking the GUS sequence with a HPR fragment in the antisense orientation upstream of the GUS and in the sense orientation on the downstream side of GUS. The pBI121 vector was digested with SmaI and SacI, the GUS sequence and the vector fragments were purified from one another. The isolated GUS fragment was digested using EcoRV and the 1079 bp blunt ended EcoRV/SacI fragment isolated. This was ligated back into the digested parent vector at the SmaI/SacI site. The HPR fragments to be cloned in opposite orientations were produced using the primer pairs identified by SEQ ID NO:8 and SEQ ID NO:11, to produce the antisense orientation and SEQ ID NO:12 and SEQ ID NO:13, to produce the sense orientation fragment. These primers incorporated restriction sites advantageous for the cloning strategy. The sense fragment was inserted at the SacI site and the antisense fragment between the XbaI and BamHI sites.

The HPR promoter was PCR amplified from genomic DNA isolated from wild type Arabidopsis thaliana (ecotype Columbia) using primers which were designed based on sequence data in Accession number AC012563. The primer pair identified by SEQ D NO:14 and SEQ ID NO:15 were used to PCR amplify the promoter and the first 2 codons of the HPR gene. The DNA fragment was cloned into pbluescript T/A vector at the EcoRV site and sequenced. The fragment was cloned into pBI121 at the HindIII and BamHI sites, replacing the 35S promoter of that plasmid. A truncated version of the promoter was produced using the primer pair identified by SEQ ID NO:16 and SEQ ID NO:15 and cloned as above. The resulting plasmids are referred to as pHPR-GUS and pHPRT-GUS respectively.

### EXAMPLE 4

### Sequence Analysis

A disclosed nucleic acid of 1161 nucleotides (SEQ ID NO:1) and also referred to as HPR, is shown in Table 1

TABLE 1A
HPR Nucleotide Sequence (SEQ ID NO:1).

ATGGCGAAACCGGTGTCCATTGAAGTGTATAATCCTAATGGGAAATACAG
AGTTGTTAGCACAAAACCGATGCCTGGAACTCGCTGGATCAATCTCTTGG
TAGACCAAGGTTGTCGCGTTGAGATATGTCATTTGAAGAAGACATCTTGT
CTGTAGAAGATATCATTGATCTGATCGGAGACAAGTGTGATGGAGTCATC
GGTCAGTTGACGGAAGATTGGGGAGAGACTCTGTTCTCAGCTTTGAGCAA
AGCTGGAGGGAAAGCTTTCAGTAACATGGCCGTTGGTTATAACAACGTTG
ATGTTGAAGCTGCCAATAAGTATGGAATTGCTGTCGGTAACACTCCGGGA
GTGTTGACTGAGACGACGGCTGAACTAGCTGCTTCTCTTTCCTTGGCTGC
TGCAAGAGAAGAATTGTTGAAGCCGACGAATTCATGAGAGGTGGCTTGTA
CGAGGGATGGCTTCCTCATCTGTTTGTGGGGAACTTACTTAAAGGACAGA
CTGTTGGAGTTATTGGAGCTGGACGTATTGGATCTGCTTATGCTAGAATG
ATGGTGGAAGGGTTCAAGATGAATTTGATCTACTTTGATCTTTACCAATC
CACTCGTCTTGAGAAATTTGTGACAGCTTATGGACAGTTCTTGAAAGCAA
ATGGAGAACAACCTGTGACATGGAAACGAGCTTCGTCCATGGAGGAGGTG
CTGCGTGAGGCTGATCTGATAAGTCTTCACCCGGTGCTGGACAAAACCAC
TTACCATCTTGTCAACAAGGAGAGGCTTGCCATGATGAAAAAGGAAGCAA
TCCTTGTGAACTGCAGCAGAGGTCCTGTGATCGATGAGGCAGCTTTGGTC
GAACATCTCAAAGAGAACCCGATGTTCCGAGTTGGTCTCGATGTGTTCGA
GGAAGAGCCATTCATGAAACCAGGGCTTGCTGATACGAAAAACGCTATTG
TTGTTCCTCACATTGCTTCTGCTTCCAAGTGGACTCGTGAAGGAATGGCT
ACGCTTGCAGCTCTCAACGTCCTCGGAAGAGTCAAAGGGTACCCGATTTG
GCATGACCCGAACCGAGTCGATCCATTCTTGAACGAAAACGCTTCACCGC
CCAATGCCAGTCCAAGCATCGTCAACTCAAAGGCCTTAGGATTGCCTGTT
TCGAAGCTATGA

A disclosed HPR polypeptide (SEQ ID NO:2) encoded by SEQ ID NO:1 has 386 amino acid residues and is presented in Table 1B using the one-letter amino acid code.

TABLE 1B
Encoded HPR protein sequence (SEQ ID NO:2).

MAKPVSIEVYNPNGKYRVVSTKPMPGTRWINLLVDQGCRVEICHLKKTIL
SVEDIIDLIGDKCDGVIGQLTEDWGETLFSALSKAGGKAFSNMAVGYNNV
DVEAANKYGIAVGNTPGVLTETTAELAASLSLAARRIVEADEFMRGGLYE
GWLPHLFVGNLLGQTVGVIGAGRIGSAYARMMVEDGKMNLIYFDLYQSTR
LEKFVTAYGQFLKANGEQPVTWKRASSMEEVLREADLISLHPVLDKTTYH
LVNKERLAMMKKEAILVNCSRGPVIDEAALVEHLKENPMFRBGLDVFEEE
PFMKPGLADTKNAIVVPHIASASKWTREGMATLAALNVLGRVKGYPIWHD
PNRVDPFLNENASPPNASPSIVNSKALGLPVSKL

The present invention also includes a nucleic acid sequence complimentary to the Arabidopsis thaliana HPR of SEQ ID NO: 1. The disclosed complimentary sequence is shown as SEQ ID NO:3.

TABLE 1C

Nucleotide Sequence Complimentary to HPR
(SEQ ID NO:3).

TCATAGCTTCGAAACAGGCAATCCTAAGGCCTTTGAGTTGACGATGCTTG
GACTGGCATTGGGCGGTGAAGCGTTTTCGTTCAAGAATGGATCGACTCGG
TTCGGGTCATGCCAAATCGGGTACCCTTTGACTCTTCCGAGGACGTTGAG
AGCTGCAAGCGTAGCCATTCCTTCACGAGTCCACTTGGAAGCAGAAGCAA
TGTGAGGAACAACAATAGCGTTTTTCGTATCAGCAAGCCCTGGTTTCATG
AATGGCTCTTCCTCGAACACATCGAGACCAACTCGGAACATCGGGTTCTC
TTTGAGATGTTCGACCAAAGCTGCCTCATCGATCACAGGACCTCTGCTGC
AGTTCACAAGGATTGCTTCCTTTTTTCATCATGGCAAGCCTCTCCTTGTT
GACAAGATGGTAAGTGGTTTTGTCCAGCACCGGGTGAAGACTTATCAGAT
CAGCCTCACGCAGCACCTCCTCCATGGACGAAGCTCGTTTCCATGTCACA
GGTTGTTCTCCATTTGCTTTCAAGAACTGTCCATAAGCTGTCACAAATTT
CTCAAGACGAGTGGATTGGTAAGATCAAAGTAGATCAATTCATCTTGAAC
CCTTCCACCATCATTCTAGCATAAGCAGATCCAATACGTCCAGCTCCAAT
AACTCCAACAGTCTGTCCTTTAAGTAAGTTCCCCACAAACAGATGAGGAA
GCCATCCCTCGTACAAGCCACCTCTCATGAATTCGTCGGCTTCAACAATT
CTTCTTGCAGCAGCCAAGGAAAGAGAAGCAGCTAGTTCAGCCGTCGTCTC
AGTCAACACTCCCGGAGTGTTACCGACAGCAATTCCATACTTATTGGCAG
CTTCAACATCAACGTTGTTATAACCAACGGCCATGTTACTGAAAGCTTTC
CCTCCAGCTTTGCTCAAAGCTGAGAACAGAGTCTCTCCCCAATCTTCCGT
CAACTGACCGATGACTCCATCACACTTGTCTCCGATCAGATCAATGATAT
CTTCTACAGACAAGATTGTCTTCTTCAAATGACATATCTCAACGCGACAA
CCTTGGTCTACCAAGAGATTGATCCAGCGAGTTCCAGGCATCGGTTTTGT
GCTAACAACTCTGTATTTCCCATTAGGATTATACACTTCAATGGACACCG
GTTTCGCCAT

Arabidopsis tiacliana HPR Promoter

A disclosed nucleic acid of 512 nucleotides (SEQ ID NO:4) and also referred to as HPR promoter, is shown in Table 2A.

TABLE 2A
HPR Promoter Sequence (SEQ ID NO:4).

GAAGCAGCAGAAGCCTTGATCATCTTCCTTTGTCTCAACCTGAAACTCTT
TTTTTTCTTTCATTGTTTGTTCTCTTTTCACTGTGGATGTAGATAATTGT
TTTTAATGAAATGAAGAAATATTGATTTGCCTTTTGACATAATTTTGTTA
ATAATCTTGATTACAAATTTTAGTCAGTGTTTGATGCATAGTTGCATACT
GCAGAGTTGAGTTTGGATATGGCCACGTCAGCATTATCTCGTTACCAAAA
CGTAAGGTCCAAACTCAGATAATACAAACGAAGCAGTTCTTTGTCACTCT
ATCATCAACATATGAACCACACCAAAAAAGAACAAAATCGTAGATAATGA
TCATGCAAAACCGACCGTTGGATCTTACTTTCGATTTCAAACCACATAAA
TCTTAGTGACTGAGCTAAAAAACTGAAATTTTTTAAAAGGCAAGACCTCC
TCTGTTTCCATATTCTCACCACAGAAGAACTCTTGAGGCTTTCTCTTTTC
TCTACCATGGCG

A disclosed nucleic acid of 288 nucleotides (SEQ ID NO:5) and also referred to as HPR truncated promoter (HPRT), is shown in Table 2B.

TABLE 2B
HPR Truncated Promoter Sequence (SEQ ID NO:5).

ACGTCAGCATTATCTCGTTACCAAAACGTAAGGTCCAAACTCAGATAATA
CAAACGAAGCAGTTCTTTGTCACTCTATCATCAACATATGAACCACACCA
AAAAAGAACAAAATCGTAGATAATGATCATGCAAAACCGACCGTTGGATC
TTACTTTCGATTTCAAACCACATAAATCTTAGTGACTGAGCTAAAAAACT
GAAATTTTTTAAAAGGCAAGACCTCCTCTGTTTCCATATTCTCACCACAG
AAGAACTCTTGAGGCTTTCTCTTTTCTCTACCATGGCG

### EXAMPLE 5

### Plant Transformation

Arabidopsis transgenic plants were made by the method of dipping flowering plants into an Agrobacterium culture, based on the method of Andrew Bent in, Clough S J and Bent A F, 1998. Floral dipping: a simplified method for Agrobacterium-mediated transformation of Arabidopsis thaliana. Wild type plants were grown under standard conditions with a 16 hour, 8 hour light to dark day cycle, until the plant has both developing flowers and open flowers. The plant was inverted for 2 minutes into a solution of Agrobacterium culture carrying the appropriate gene construct. Plants were then left horizontal in a tray and kept covered for two days to maintain humidity and then righted and bagged to continue growth and seed development. Mature seed was bulk harvested.

Transformed T1 plants were selected by germination and growth on MS plates containing 50 μg/ml kanamycin. Green, kanamycin resistant (Kan^{R}) seedlings were identified after 2 weeks growth and transplanted to soil. Plants were bagged to ensure self fertilization and the T2 seed of each plant harvested separately. During growth of T1 plants leaf samples were harvested, DNA extracted and Southern blot and PCR analysis performed.

T2 seeds were analyzed for Kan^{R} segregation. From those lines that showed a 3:1 resistant phenotype, surviving T2 plants were grown, bagged during seed set, and T3 seed harvested from each line. T3 seed was again used for Kan^{R} segregation analysis and those lines showing 100% Kan^{R} phenotype were selected as homozygous lines. Further molecular and physiological analysis was done using T3 seedlings.

Transgenic Brassica napus, Glycine max and Zea maize plants can be produced using Agrobacterium mediated transformation of cotyledon petiole tissue. Seeds are sterilized as follows. Seeds are wetted with 95% ethanol for a short period of time such as 15 seconds. Approximately 30 ml of sterilizing solution I is added (70% Javex, 100 μl Tween20) and left for approximately 15 minutes. Solution I is removed and replaced with 30 ml of solution II (0.25% mecuric chloride, 100 μl Tween20) and incubated for about 10 minutes. Seeds are rinsed with at least 500 ml double distilled sterile water and stored in a sterile dish. Seeds are germinated on plates of ½ MS medium, pH 5.8, supplemented with 1% sucrose and 0.7% agar. Fully expanded cotyledons are harvested and placed on Medium I (Nurashige minimal organics (MMO), 3% sucrose, 4.5 mg/L benzyl adenine (BA), 0.7% phytoagar, pH5.8). An Agrobacterium culture containing the nucleic acid construct of interest is grown for 2 days in AB Minimal media. The cotyledon explants are dipped such that only the cut portion of the petiole is contacted by the Agrobacterium solution. The explants are then embedded in Medium I and maintained for 5 days at 24° C., with 16,8 hr light dark cycles. Explants are transferred to Medium II (Medium I, 300 mg/L timentin,) for a further 7 days and then to Medium III (Medium II, 20 mg/L kanamycin). Any root or shoot tissue which has developed at this time is dissected away. Transfer explants to fresh plates of Medium III after 14-21 days. When regenerated shoot tissue develops the regenerated tissue is transferred to Medium IV (MMO, 3% sucrose, 1.0% phytoagar, 300 mg/L timentin, 20 mg/L 20 mg/L kanamycin). Once healthy shoot tissue develops shoot tissue dissected from any callus tissue are dipped in 10×IBA and transferred to Medium V (Murashige and Skooge (MS), 3% sucrose, 0.2 mg/L indole butyric acid (D3A), 0.7% agar, 300 mg/L timentin, 20 mg/L 20 mg/L kanamycin) for rooting. Healthy plantlets are transferred to soil. The above method, with or without modifications, is suitable for the transformation of numerous plant species including Glycine max, Zea maize and cotton.

Transgenic Glycine max, Zea maize and cotton can be produced using Agrobacterium-based methods which are known to one of skill in the art. Alternatively one can use a particle or non-particle biolistic bombardment transformation method. An example of non-particle biolistic transformation is given in U.S. Patent Application 20010026941. This method has been used to produce transgenic Glycine max and Zea maize plants. Viable plants are propagated and homozygous lines are generated. Plants are tested for the presence of drought tolerance, physiological and biochemical phenotypes as described elsewhere.

Transformation of plant tissue such as Zea maize for example, can be achieved by sonication of callus tissue culture. Callus tissue was produced as follows. Ears of corn were harvested 18 days after silking and surface sterilized in 50% v/v bleach for 20 minutes followed by three washing with sterile distilled water. Immature embryos ranging in size from 2 to 4 mm were harvested from the kernels. Embryos were placed on MSD_{1.5} medium (2% sucrose, 1×MS macronutrient and micronutrient salts, 1×MS vitamins, 1.5 mg / L 2,4-D, 0.8% agar, pH 5.8) scutellum side up. Embryos were incubate at 26-28° C. in the dark. Friable callus from 2 week old cultures were transferred to fresh MSD_{1.5} medium and further incubated at 26-28° C. in the dark. Friable callus was subcultured to fresh MSD_{1.5} medium every 21 days.

Transformation of callus tissue was performed as described below. The construct was introduced into GV3101 Agrobacterium by inoculation of a single colony of GV3101 Agrobacterium containing the HPR-GUS plasmid into 10 mL of LB amended with 150 μg/mL rifampicin, 100 μg/mL gentamycin sulfate, and 50 μg/mL kanamycin. The culture was allowed to grow overnight at 28° C. with 200 rpm shaklng. Corn callus was cut into pieces approximately 3-5 mm in size. The Agrobacterium culture was centrifuged at 1500×g for 10 minutes and washed twice with 10 mL liquid MSD_{1.5} liquid (2% sucrose, 1×MS macronutrient and micronutrient salts, 1×MS vitamins, 1.5 mg/L 2,4-D, pH 5.8). The bacteria was resuspended in liquid MSD_{1.5} to an OD_{600 nm} of 0.25 and 1 mL of diluted Agrobacterium or liquid MSD_{1.5}, for negative controls, was placed in 1.5 mL microfuge tubes containing four pieces of callus added to each tube. Callus and Agrobacterium culture was sonicated in a Branson 200 Ultrasonic Cleaner for 0, 3, 10, 30, 100, or 300 seconds with bacteria or 0 or 300 seconds without bacteria (in MSD_{1.5} liquid alone). After sonication, the callus was blotted on sterile filter paper and placed on MSD_{1.5}A medium (MSD_{1.5} solid medium amended with 100 μM acetosyringone). The co-cultivation period was 4 days in the dark at 28° C. Callus was rinsed in liquid MSD_{1.5}, blotted on sterile filter paper, and placed on MSD_{1.5}T medium (MSD_{1.5} solid medium amended with 400 μg/mL Timentin) for 3 days in the dark at 28° C. Seven days after sonication, callus was added to 1 mL GUS staining solution (50 mM NaPO₄, pH 7.0, 0.1% Triton X-100, 1 mM EDTA, 2 mM DTT, 0.5 mg/mL X-GlcA) and left to incubate overnight at 37° C. The staining solution was replaced with 1 mL fixation buffer (10% formaldehyde, 50% ethanol) and incubated for 30 minutes at room temperature. The fixation buffer was replaced with 80% ethanol and incubated for 1 hour at room temperature. The 80% ethanol was replaced with 100% ethanol and incubated for 1 hour at room temperature. The callus was assessed for blue staining, indicating GUS activity.

### EXAMPLE 6

### Assessment of ABA Sensitivity

Approximately 100 seeds were assessed per line per 9 cm plate. Seeds were sterilized with 50% bleach for 8 minutes and washed four times with sterile water. Seeds were plated on minimal medium (½ MS salt, without sucrose and vitamins) supplemented with no ABA, 0.25 μM, 0.5 μM, or 1.0 μM ABA. Plates were chilled for 3 days at 4° C. in the dark, and incubated for up to 21 days at 22° C. with 16 hour, 8 hour light to dark light cycle. Plates were assessed for germination, cotyledon expansion, true leaf development and seedling vigor. Seedlings were assessed for ABA sensitivity over 21 days of growth at which time sensitive seedlings were arrested at the cotyledon stage, lacked true leaves, and showed inhibition of root growth. Wild type control Columbia plants had two to three pairs of true leaves and a well developed root system. Lines were categorized as ABA sensitive (ABA^{S}) if less than 1% of plants looked like control, moderately ABA sensitive (ABA^{MS}) if more than 1% but less than 50% of looked like control, or ABA insensitive (ABA^{Wt}) if greater than 50% looked like control. For example, if a plate had 20 healthy seedlings and the control plate had 60 healthy seedlings, the line would be 33% of control and categorized as moderately ABA sensitive.

All three vector constructs (pRD29A-HPR, pBI121-HPR AND pBI121-hp-HPR) have resulted in transgenic lines of Arabidopsis which have increased sensitivity to ABA which is indicative of stress tolerance. Nineteen lines of pRD29A-HPR were ABA^{S} at 0.5 μM ABA, seven pBI121-HPR lines and three pBI121-hp-HPR lines showed ABA^{S} characteristics.

### EXAMPLE 7

### Southern Blot Analysis

Genomic Southern blot analysis of transgenic Arabidopsis was performed using standard techniques known to one skilled in the art. Typically, 5 μg of DNA was electrophoresed in a 0.8% agarose gel and transferred to an appropriate membrane such as Hybond N+ (Amersham Pharmacia Biotech). Pre-hybridization and hybridization conditions were as suggested by the membrane manufacturer, typically at 65° C. The final stringency wash was typically at 1×SSC and 0.1% SDS at 65° C. The NPTII coding region was typically used as the radiolabeled probe in Southern blot analysis. Transgenic Arabidopsis lines were selected as homozygous based on segregation patterns observed from NPTII probed Southern blots. Lines were confirmed to be transgenic by PCR analysis using transgene specific primers in the PCR assays. Lines of pBI121-HPR were confirmed using the primer pair identified by SEQ ID NO:27 and SEQ ID NO:7. Lines of pRD29A-HPR were confirmed using the primer pair identified by SEQ ID NO:9 and SEQ ID NO:7. Lines of pBI121-hp-HPR were confirmed using the primer pair identified by SEQ ID NO:27 and SEQ ID NO:8.

### EXAMPLE 8

### Northern Blot Analysis

Total RNA was isolated from transgenic and wild type Arabidopsis lines (T3 plants). Approximately 10 μg of total RNA was loaded into each lane. The Northern was probed with radiolabeled HPR cDNA in ExpressHyb hybridization solution (Clontech) and exposed using a phosphoimaging screen. For quantification blots were reprobed with tubulin, a constitutively expressed gene, for a comparative standard.

A survey of HPR expression in various tissues of wild type plants shows that expression is detectable in aerial tissues but not in roots.

The endogenous HPR expression level in wild-type Arabidopsis leaves during drought stress treatment was examined. Plants were grown under optimal conditions in the growth chamber for three weeks before withholding water to mimic a drought stress. Leaf samples were collected for total RNA extraction at day 0, 2, 4, and 6 after cessation of watering. The level of HPR gene expression increased over the water stress, to a maximum expression level, which was 60% greater than initial, as of day 4 of the drought stress. This demonstrates the up-regulation of Arabidopsis HPR expression by an abiotic stress such as drought.

Northern analysis shows that the HPR expression found in Line 28.1 is approximately half of that found in wild type plants. Thereby providing further data that HPR is the affected gene in Line 28.1 PIS1 expression was the same in line 28.1 as in the wild type control.

Transgenic pBI121-HPR lines were assessed for HPR expression by Northern analysis. Thirteen of eighteen lines examined showed an increase in gene expression, typically by two or three fold but also to as much as fourteen fold increase. The lines that showed altered HPR expression correlated with the lines that showed increased ABA sensitivity.

Transgenic pBI121-hp-HPR lines were assessed for HPR expression by Northern analysis. Seven lines examined showed a decrease in gene expression with two lines having undetectable expression. The lines that showed altered HPR expression correlated with the lines that showed increased ABA sensitivity.

Transgenic pRD29A-HPR lines are assessed for HPR expression by Northern analysis. The pRD29A promoter is a drought inducible promoter and as such Northern analysis is conducted after plants have been exposed to a drought stress treatment.

### EXAMPLE 9

### HPR Promoter Analysis

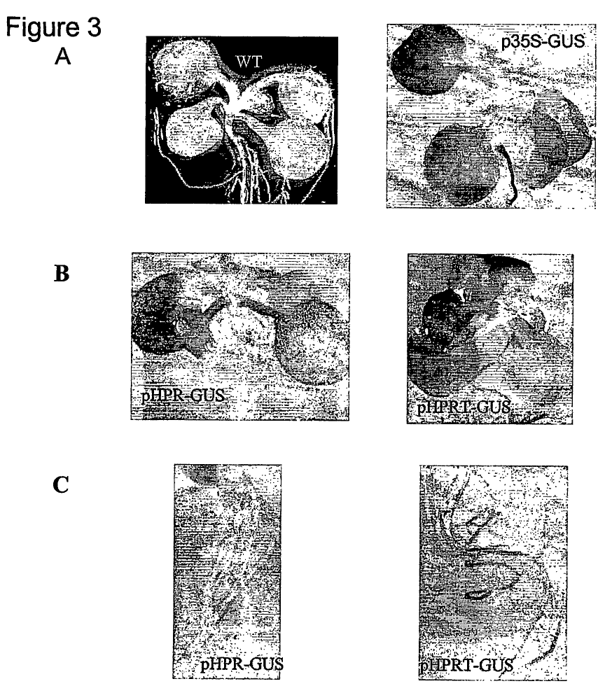

Transgenic Arabidopsis lines of pHPR-GUS and pHPRT-GUS were analyzed for GUS staining activity to determine the expression characteristics of the isolated promoter fragments. One week old seedlings were incubated at 37° C. in staining buffer (50 mM NaPO₄ pH 7.0, 10 mM EDTA, 0.1% Triton X-100, 0.5 mg/mL X-Gluc) for 16 hours. Wild type control plants showed no GUS staining while transgenic plants having the pHPR-GUS construct showed staining in aerial tissue of young seedlings (FIG. 3B) but no detectable expression in roots (FIG. 3C). Analysis of flowers showed GUS activity particularly in the petals and stigma tissue. Comparison of pHPR-GUS transgenic plants to those harboring the p121-GUS construct containing the 35S CaMV promoter shows that the intensity of expression by the HPR promoter is equivalent to that of the 35S promoter (FIGS. 3A,B).

Analysis of pHPRT-GUS lines did show expression in the roots in addition to the staining of the aerial tissues (FIG. 3C). The strength and aerial expression patterns of the pHPRT-GUS construct is equivalent to the pHPR-GUS construct.

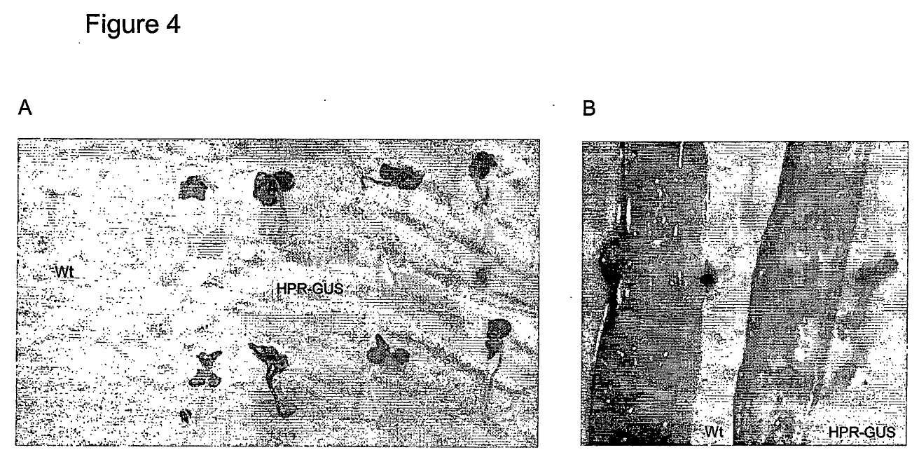

The pHPR-GUS and pHPRT-GUS promoters were evaluated in Brassica napus (canola) transgenic plants. The patterns observed in Arabidopsis were again seen in the canola plants (FIG. 4A). Analysis of the siliques and developing seeds showed expression in these tissues (FIG. 4B).

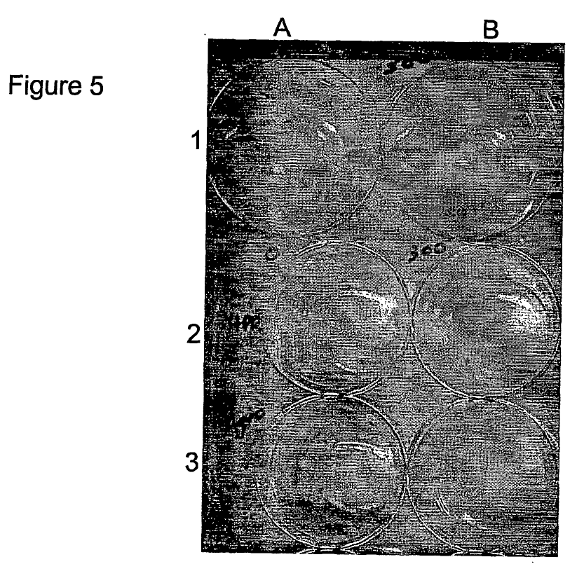

The pHPR-GUS promoter was evaluated in Zea maize (corn) transgenic callus tissue. The construct produced GUS activity in transformed corn callus tissue (FIGS. 5, 1A,1B). Negative controls did not produce GUS activity.

### EXAMPLE 10

### HPR Activity Assay

HPR enzymatic activity was assessed using a biochemical assay, an example of such an assay is as follows. Leaf tissue was ground in extraction buffer (25 mM Tris pH 7.5, 0.2 mM EDTA, 0.5 mM PMSF, 0.5 μg/mL leupeptin, 0.2 μg/mL pepstain A. 2 mM DTT, 1% Tritin X-100). An 19 μL aliquot of the clarified supernatant solution was added; to 196 μL of the assay solution (10 mM NADH, 150 mM hydroxypyruvate, 50 mM Tris pH 7.5). The change in absorbance was monitored spectophotometrically at 340 nm.

### EXAMPLE 11

### Production of Polyclonal Antibodies Against HPR

Anti-AtHPR antibodies were generated using AtHPR fusion protein over-expressed in E. coli. The over-expression vector, pMAL-p2, contains 1175 bp malE gene that is located upstream of AtHPR and encodes a 43 KDa maltose-binding protein (MBP). The AtHPR fragment to be used was PCR amplified using primer pair identified by SEQ ID NO:28 and SEQ ID NO:29. The 1161 bp XbaII/SalI DNA fragment of AtHPR was inserted into pMAL-p2 at XbaII and SalI sites. The SalI site was converted into blunt end using Klenow fragment. The resulting fusion protein MBP-AtHPR was then over-expressed in DH5α, and purified by one-step annnity for MBP as described by the manufacturer (New England Biolab). The soluble fraction of the crude bacterial extract containing the MBP-AtHPR fusion protein was loaded to a amylose column (1.5 cm×10.0 cm), and the proteins were eluted with 10 mM maltose in column buffer (50 mM Tris-HCl, pH 7.5, 1 mM EDTA, and 200 mM NaCl). Fractions containing purified MBP-AtHPR fusion protein were pooled, and concentrated with a Centriprep-30 concentrator (Amicon). All purification steps were carried out at 4° C. To generate an antibody, the purified fusion protein was further separated by SDS-PAGE and the Coomassie stained band corresponding to the fusion protein was excised. The identity of the fusion protein was confirmed by Western analysis using anti-MBP antibodies (purchased from New England Biolab). The protein was eluted from the gel slice by electroelution and then emulsified in Ribi adjuvant (Ribi Immunochem) to a final volume of 1 ml. MBP-AtHPR protein was injected into a 3 kg New Zealand rabbit on day 1 and booster injections were given on day 21 and day 35 with 250 μg of the protein each time. High-titer antisera were obtained one week after the final injection.

### EXAMPLE 12

### Western Blot Analysis of HPR Transgenic Lines Using Anti-AtHPR Antibodies

Western analysis was performed to examine expression level of AtHPR in the transgenic lines compared with that of wild type plants. Anti-Bip antibody, an ER lumenal protein (Stressgen, Victoria, BC, Canada) was used as a reference. Total proteins were extracted from developing leaf tissue of ABA^{S} lines and a wild type control. The antigenic protein bands of AtHPR and Bip were scanned and quantified using the UN-Scan-It program (Silk Scientific, Utah, USA) and the ratio of the two protein bands for each sample was obtained.

### EXAMPLE 13

### Physiology Analysis, Drought

Transgenic lines of Arabidopsis plants carrying the pBI121-HPR and pRD29A-HPR constructs were analyzed for drought tolerant phenotype characteristics. Performance of the transgenic lines was compared to that of Columbia wild type controls.

Seeds were germinated on agar plates containing ½ MS, 1% sucrose, 5 mM MES and vitamins. After one week, seedlings were transplanted into 3″ pots, five seedlings per pot, with consistent amounts of soil. Six replicate pots were planted per line. Plants were grown under optimal conditions (16 hr light at 200 uE, 70% RH, and 22C), watered daily until the first open flower. The drought treatment was initiated by watering up the pots to equal pot weight followed by cessation of further watering. Pots were weighed daily for four days at which time rosette leaves, and stems were harvested for fresh and dry weight determinations. Daily soil water content, and final plant turgidity (ratios of fresh to dry weight) were calculated. In addition, water lost in first two days/final leaf dry biomass and water lost in 2 days/final total shoot dry biomass were determined.

Drought tolerant lines were identified as lines which had greater soil water content, lower water loss per dry biomass and equal or greater dry biomass. They visually looked more turgid than controls and this observation was confirmed by greater fresh to dry weight ratios.

Transgenic lines of pBI121-HPR were advanced to the T3 generation and confirmed to be homozygous. Lines were assessed for days to first flower, water loss per day as a percentage of initial, water loss after two days drought treatment relative to biomass and fresh and dry weights of leaves and stems. Five lines were identified that demonstrated a drought tolerant phenotype.

Seven lines had significantly higher soil water content but three of those lines also had reduced biomass. Water lost in 2d/g of leaf dry weight was significantly greater for eight of the lines eliminating these as drought tolerant candidates. Three lines had significantly reduced water loss/g leaf dry weight compare to Columbia. Water lost in 2 d/g shoot dry weight was significantly greater for six lines and no lines had significantly lower water loss/shoot DW. Four of the lines had significantly higher rosette leaf DW and most of the other lines were significantly reduced in leaf DW as compared to Columbia. Stem DW was significantly reduced in four of the lines and the rest of the lines were not different from Columbia. Therefore the overall shoot DW was significantly reduced in ten lines. There were a number of significant differences in FW/DW ratios for both, stem and leaf as well as shoot but these were a reflection of smaller plants using less water. Five of the lines flowered significantly earlier, by up to two days earlier, than Columbia.

Transgenic lines of pRD29A-HPR were advanced to the T3 generation and confirmed to be homozygous. Lines were assessed for days to first flower, water loss per day as a percentage of initial, water loss after two days drought treatment relative to biomass and fresh and dry weights of leaves and stems. Eighteen lines were identified that demonstrated a drought tolerant phenotype.

Eight lines showed delayed flowering by up to two days. Most of the lines showed significantly increased soil water content, and all except one line showed significantly reduced water lost in 2 d/g leaf dry weight. Water lost in 2 d/g shoot dry weight was also significantly reduced in the majority of lines. All lines, except one, had significantly reduced stem dry weight and significantly increased rosette leaf dry weight. Additionally, stem, leaf and shoot fresh to dry weight ratios were significantly increased in many lines indicating higher water content and turgidity of these lines.

Transgenic lines of pBI121-hp-HPR were advanced to the T3 generation and confirmed to be homozygous. Lines are assessed for days to first flower, water loss per day as a percentage of initial, water loss after two days drought treatment relative to biomass and fresh and dry weights of leaves and stems.

### EXAMPLE 14

### Physiology Analysis, Photo-Oxidative Stress

Growth of transgenic lines of pBI121-hp-HPR using a 24 hour light regime resulted in plants that were under visible stress. Leaves were chlorotic and plants were generally smaller and less vigorous than wild type controls. Growth of these plants using a 16 hour light, 8 hour dark cycle relieved the symptoms of stress and plants appeared to grow normally.

### REFERENCES

Mano, S., Hayashi, M., and Nishimura, M. (1999) Light regulates alternative splicing of hydroxypyruvate reductase in pumkin. The Plant Journal. 17, 309-320.

Mano, S., Hayashi, M., Kondo, M., and Nishimura, M. (1997) Hydroxypyruvate reductase with a carboxy-terminal targeting signal to microbodies in expressed in arabidopsis. Plant Cell Physiol. 38, 449-455.

Kleczkowski, L. A., Edwards, G. E., Blackwell, R. D., Lea, P. J., and Givan, C. V. (1990) Enzymology of the reduction of hydroxypyruvate and glyoxylate in a mutant of barley lacking peroxisomal hydroxypyravate reductase. Plant Physiology. 94, 819-825.

Daniel, S. G., and Becker, W. M. (1995) Transgenic analysis of the 5′- and 3′-fk·anking regions of the NADH-dependent hydroxypyrnvate reductase gene form cucumis sativus L. Plant Molecular Biology. 28, 821-836.

Jin, G., Davey, M. C., Ertl, J. R., Chen, R., Yu, Z., Daniel, S. G., Becker, W. M., and Chen, C. (1998) Interaction of DNA-binding proteins with the 5′-fanking region of a cytokinin-responsive cucumber hydroxypyruvate reductase gene. Plant Molecular Biology. 38, 713-724.

Olsen, L., (1998) The surprising complexity of peroxisome biogenesis. Plant Molecular Biology. 38, 163-189.

Wingler, A., Quick, W. P., Bungard, R. A., Bailey, K. J., Lea, P. J., and Leegood, R. C. (1999) The role of photorespiration during drought stress: an analysis utilizing barley mutants with reduced activities of photorespiratory enzymes. Plant, Cell and Environment. 22, 361-375.

Wingler, A., Lea, P. J., Quick, W. P., and Leegood, R. C. (2000) Photorespiration: metabolic pathways and their role in stress protection. Phil. Trans. R. Soc. Lond. B. 355, 1517-1529.

Corpas, F. J., Barroso J. B., and Del Rio, L. A. (2001) Peroxisomes as a source of reactive oxygen species and nitric oxide signal molecules in plant cells. Trends in Plant Science. 6, 145-150.

Hu, J., Aguirre, M., Peto, C., Alonso, J., Ecker, J., and Chory, J. (2002) A role for peroxisomes in photomorphogenesis and development of arabidopsis. Science. 297, 405-409.

Seo, M., and Koshiba, T. (2002) Complex regulation of ABA biosynthesis in plants. Trends in Plant Science. 7, 41-48.

Hirschberg, J. (2001) Carotenoid biosynthesis in flowering plants. Current Opinion in Plant Biology. 4, 210-218.

Cunningham, F. X., and Gantt, E. (1998) Genes an enzymes of carotenoid biosynthesis in plants. Annu. Rev. Plant Physiol. Plant Mol. Biol. 49, 557-583.

Yamaguchi, K., and Nishimura, M. (2000) Reduction to below threshold levels of glycolate oxidase activities in transgenic tobacco enhances photoinhibition during irradiation. Plant Cell Physiol. 41, 1397-1406.

Mano, S., Hayashi, M., and Nishimura, M. (2000) A leaf-peroxisomal protein, hydroxypyrnvate reductase, is produced by light-regulated alternative splicing. Cell Biochein Biophys. 32, 147-154.

Sloan J. S., Schwartz and Becker, The Plant Journal (1993) 3(6): 867-874

Daniel S. G., Becker, Plant Mol. Biol. (1995) 28: 821-836

Mano S., Hayashi, Kondo, Nishimura, Plant Cell Physiol. (1997) 38(4): 449-455

Jin G., Davey, Ertl, Chen, Yu, Daniel, Becker, Chen, Plant Mol. Biol. (1998) 38: 713-724

Mano S., Hayashi, Nishimura, The Plant Journal (1999) 17(3): 309-320

Schwartz, Sloan Becker, Accession No.: X58542 PMB (1991) 17(4) 941-947

Lin, X., et al. (1991) Accession No.: AC012563 gi:12324062 15 Jun. 2001

Greenler J. M., Sloan J. S, Schwartz B. W., Becker W. M. Plant Molecular Biology, (1989) 13: 139-150

Greenler J. M. and Becker W. M, Plant Physiology (1990) 94: 1484-1487

Hayashi M., Tsugeki R., Kondo M., Mori H., Nishimura M., Plant Molecular Biology (1996) 30: 183-189

Hondred D., Wadle D-M., Titus D. E., Becker W. M., Plant Molecular Biology (1987) 9: 259-275

Wingler, A., Quick, W. P., Bungard R. A., Bailey K. J., Lea, P. J., and Leegood, R. C. Pant Cell and Environment (1999) 22:361-373.

1. An isolated nucleic acid molecule comprising the nucleotide sequence of SEQ ID NO:4 or SEQ ID NO: 5 wherein said nucleic acid molecule is less than 1000 nucleotides in length.

2. The isolated nucleic acid molecule of claim 1, wherein said nucleic acid molecule is less than 800 nucleotides in length.

3. The isolated nucleic acid molecule of claim 1, wherein said nucleic acid molecule is less than 750 nucleotides in length.

4. The isolated nucleic acid molecule of claim 1, wherein said nucleic acid molecule is less than 600 nucleotides in length.

5. An isolated nucleic acid molecule consisting of the nucleotide sequence of SEQ ID NO:4 or fragment thereof.

6. An isolated nucleic acid molecule consisting of the nucleotide sequence of SEQ ID NO:5 or fragment thereof.

7. The isolated nucleic acid molecule of claim 1, wherein said sequence regulates transcription of an operably linked nucleotide sequence of interest.

8. The isolated nucleic acid molecule of claim 7, wherein said sequence regulates transcription by inducing expression in response to a stimulus.

9. The isolated nucleic acid molecule of claim 8, wherein said stimulus is light or an environmental stress.

10. A isolated nucleic acid construct comprising, a promoter sequence comprising the nucleic acid sequence of SEQ ID NO: 4 or 5 or fragment thereof operably linked to a nucleotide sequence encoding a heterologous gene, wherein said heterologous gene encodes a protein of interest or fragment thereof.

11. The construct of claim 10, wherein said construct comprises at least two promoter sequences.

12. The construct of claim 11, further comprising a spacer sequence, wherein said spacer sequence operably links said promoter sequences.

13. The construct of claim 10, further comprising a nucleic acid encoding a selectable marker.

14. The construct of claim 10, further comprising a nucleic acid encoding a reporter gene.

15. The construct of claim 10, wherein said heterologous gene is capable of altering an agronomic trait.

16. The construct if claim 15, wherein said agronomic trait is disease resistance, herbicide resistance, environmental stress resistance, enhanced growth, or increased yield.

17. The construct of claim 10, wherein said heterologous gene is a plant gene.

18. The construct of claim 10, wherein said heterologous gene is a structural gene.

19. The construct of claim 18, wherein said structural gene is an enzyme, a transcriptional regulator, a chaperonin protein or a scaffolding protein.

20. The construct of claim 19, wherein said enzyme is farnesyl transferase alpha, farnesyl transferase beta or CaaX prenyl protease.

21. A isolated nucleic acid construct comprising, a promoter sequence comprising SEQ ID NO: 4 or 5 or fragment thereof operably linked to a non-translatable mRNA molecule of a gene encoding a protein of interest.

22. The construct of claim 21, wherein said non-translated mRNA molecule is an antisense nucleic acid, a hairpin RNA or a microRNA.

23. The construct of claim 21, fuirther comprising a nucleic acid encoding a selectable marker.

24. The construct of claim 21, further comprising a nucleic acid encoding a reporter gene.

25. The construct of claim 21, wherein said gene is capable of altering an agronomic trait.

26. The construct if claim 25, wherein said agronomic trait is disease resistance, herbicide resistance, environmental stress resistance, enhanced growth or increased yield.

27. The construct of claim 21, wherein said gene is a plant gene.

28. The construct of claim 21, wherein said gene is a structural gene.

29. The construct of claim 28, wherein said structural gene is an enzyme, a transcriptional regulator, a chaperonin protein or a scaffolding protein.

30. The construct of claim 29, wherein said enzyme is farnesyl transferase alpha, farnesyl transferase beta or CaaX prenyl protease.

31. A vector comprising the nucleic acid molecule of claim 1.

32. A cell comprising the vector of claim 31.

33. The cell of claim 32, wherein said cell is a plant cell.

34. The cell of claim 33, wherein said plant cell is monocotyledonous.

35. The cell of claim 33, wherein said plant cell is dicotyledonous.

36. A vector comprising the nucleic acid construct of claim 10.

37. A cell comprising the vector of claim 36.

38. The cell of claim 37, wherein said cell is a plant cell.

39. The cell of claim 38, wherein said plant cell monocotyledonous.

40. The cell of claim 38, wherein said plant cell is dicotyledonous.

41. A vector comprising the nucleic acid construct of claim 21.

42. A cell comprising the vector of claim 41.

43. The cell of claim 42, wherein said cell is a plant cell.

44. The cell of claim 43, wherein said plant cell monocotyledonous.

45. The cell of claim 43, wherein said plant cell is dicotyledonous.

46. A method of producing a transgenic plant comprising introducing into a plant cell the vector of claim 36, to generate a transgenic cell and regenerating a transgenic plant from said transgenic cell, wherein said transgenic plant expresses said protein of interest.

47. The method of claim 46, wherein said expression is constitutive.

48. The method of claim 46, wherein said expression is inducible

49. The method of claim 46, wherein said plant cell is monocotyledonous.

50. The method of claim 46, wherein said plant cell is dicotyledonous.

51. A method of producing a transgenic plant comprising introducing into a plant cell the vector of claim 41, to generate a transgenic cell and regenerating a transgenic plant from said transgenic cell, wherein said transgenic plant expresses said protein of interest at a decreased level as compared to a wildtype plant

52. The method of claim 51, wherein said plant cell is monocotyledonous.

53. The method of claim 51, wherein said plant cell is dicotyledonous.

54. The transgenic plant produced by the method of claim 46.

55. The seed produced by the transgenic plant of claim 54, wherein said seed produces a plant that expresses said protein of interest.

56. The transgenic plant produced by the method of claim 51.

57. The seed produced by the transgenic plant of claim 56, wherein said seed produces a plant that expresses said protein of interest at a decreased level as compared to a wildtype plant.

58. A method of expressing a heterologous protein comprising introducing to a cell the construct of of claim 10 and expressing said heterologous protein in said cell.

59. The method of claim 58, wherein said cell is a plant cell.

60. The method of claim 59, wherein said plant cell is monocotyledonous.

61. The method of claim 59, wherein said plant cell is dicotyledonous.


SEQUENCE LISTING (30 sequences)

SEQ ID NO: 1 - DNA, 1161 bp, Artificial
  Hydroxypyruvate reductase (HPR) nucleic acid sequence
atggcgaaaccggtgtccattgaagtgtataatcctaatgggaaatacagagttgttagcacaaaaccgatgcctggaactcgctggatcaatctcttggtagaccaaggttgtcgcgttgagatatgtcatttgaagaagacaatcttgtctgtagaagatatcattgatctgatcggagacaagtgtgatggagtcatcggtcagttgacggaagattggggagagactctgttctcagctttgagcaaagctggagggaaagctttcagtaacatggccgttggttataacaacgttgatgttgaagctgccaataagtatggaattgctgtcggtaacactccgggagtgttgactgagacgacggctgaactagctgcttctctttccttggctgctgcaagaagaattgttgaagccgacgaattcatgagaggtggcttgtacgagggatggcttcctcatctgtttgtggggaacttacttaaaggacagactgttggagttattggagctggacgtattggatctgcttatgctagaatgatggtggaagggttcaagatgaatttgatctactttgatctttaccaatccactcgtcttgagaaatttgtgacagcttatggacagttcttgaaagcaaatggagaacaacctgtgacatggaaacgagcttcgtccatggaggaggtgctgcgtgaggctgatctgataagtcttcacccggtgctggacaaaaccacttaccatcttgtcaacaaggagaggcttgccatgatgaaaaaggaagcaatccttgtgaactgcagcagaggtcctgtgatcgatgaggcagctttggtcgaacatctcaaagagaacccgatgttccgagttggtctcgatgtgttcgaggaagagccattcatgaaaccagggcttgctgatacgaaaaacgctattgttgttcctcacattgcttctgcttccaagtggactcgtgaaggaatggctacgcttgcagctctcaacgtcctcggaagagtcaaagggtacccgatttggcatgacccgaaccgagtcgatccattcttgaacgaaaacgcttcaccgcccaatgccagtccaagcatcgtcaactcaaaggccttaggattgcctgtttcgaagctatga

SEQ ID NO: 2 - protein, 386 aa, Artificial
  encoded HPR protein sequence
MAKPVSIEVYNPNGKYRVVSTKPMPGTRWINLLVDQGCRVEICHLKKTILSVEDIIDLIGDKCDGVIGQLTEDWGETLFSALSKAGGKAFSNMAVGYNNVDVEAANKYGIAVGNTPGVLTETTAELAASLSLAAARRIVEADEFMRGGLYEGWLPHLFVGNLLKGQTVGVIGAGRIGSAYARMMVEGFKMNLIYFDLYQSTRLEKFVTAYGQFLKANGEQPVTWKRASSMEEVLREADLISLHPVLDKTTYHLVNKERLAMMKKEAILVNCSRGPVIDEAALVEHLKENPMFRVGLDVFEEEPFMKPGLADTKNAIVVPHIASASKWTREGMATLAALNVLGRVKGYPIWHDPNRVDPFLNENASPPNASPSIVNSKALGLPVSKL

SEQ ID NO: 3 - DNA, 1161 bp, Artificial
  nucleotide sequence complimentary to HPR
tcatagcttcgaaacaggcaatcctaaggcctttgagttgacgatgcttggactggcattgggcggtgaagcgttttcgttcaagaatggatcgactcggttcgggtcatgccaaatcgggtaccctttgactcttccgaggacgttgagagctgcaagcgtagccattccttcacgagtccacttggaagcagaagcaatgtgaggaacaacaatagcgtttttcgtatcagcaagccctggtttcatgaatggctcttcctcgaacacatcgagaccaactcggaacatcgggttctctttgagatgttcgaccaaagctgcctcatcgatcacaggacctctgctgcagttcacaaggattgcttcctttttcatcatggcaagcctctccttgttgacaagatggtaagtggttttgtccagcaccgggtgaagacttatcagatcagcctcacgcagcacctcctccatggacgaagctcgtttccatgtcacaggttgttctccatttgctttcaagaactgtccataagctgtcacaaatttctcaagacgagtggattggtaaagatcaaagtagatcaaattcatcttgaacccttccaccatcattctagcataagcagatccaatacgtccagctccaataactccaacagtctgtcctttaagtaagttccccacaaacagatgaggaagccatccctcgtacaagccacctctcatgaattcgtcggcttcaacaattcttcttgcagcagccaaggaaagagaagcagctagttcagccgtcgtctcagtcaacactcccggagtgttaccgacagcaattccatacttattggcagcttcaacatcaacgttgttataaccaacggccatgttactgaaagctttccctccagctttgctcaaagctgagaacagagtctctccccaatcttccgtcaactgaccgatgactccatcacacttgtctccgatcagatcaatgatatcttctacagacaagattgtcttcttcaaatgacatatctcaacgcgacaaccttggtctaccaagagattgatccagcgagttccaggcatcggttttgtgctaacaactctgtatttcccattaggattatacacttcaatggacaccggtttcgccat

SEQ ID NO: 4 - DNA, 512 bp, Artificial
  HPR promoter sequence
gaagcagcagaagccttgatcatcttcctttgtctcaacctgaaactcttttttttctttcattgtttgttctcttttcactgtggatgtagataattgtttttaatgaaatgaagaaatattgatttgccttttgacataattttgttaataatcttgattacaaattttagtcagtgtttgatgcatagttgcatactgcagagttgagtttggatatggccacgtcagcattatctcgttaccaaaacgtaaggtccaaactcagataatacaaacgaagcagttctttgtcactctatcatcaacatatgaaccacaccaaaaaagaacaaaatcgtagataatgatcatgcaaaaccgaccgttggatcttactttcgatttcaaaccacataaatcttagtgactgagctaaaaaactgaaattttttaaaaggcaagacctcctctgtttccatattctcaccacagaagaactcttgaggctttctcttttctctaccatggcg

SEQ ID NO: 5 - DNA, 288 bp, Artificial
  HPR truncated promoter sequence
acgtcagcattatctcgttaccaaaacgtaaggtccaaactcagataatacaaacgaagcagttctttgtcactctatcatcaacatatgaaccacaccaaaaaagaacaaaatcgtagataatgatcatgcaaaaccgaccgttggatcttactttcgatttcaaaccacataaatcttagtgactgagctaaaaaactgaaattttttaaaaggcaagacctcctctgtttccatattctcaccacagaagaactcttgaggctttctcttttctctaccatggcg

SEQ ID NO: 6 - DNA, 26 bp, Artificial
  HPRClaI primer
aaatcgatatggcgaaaccggtgtcc

SEQ ID NO: 7 - DNA, 29 bp, Artificial
  HPRBamHI primer
cgggatcctcatagcttcgaaacaggcaa

SEQ ID NO: 8 - DNA, 29 bp, Artificial
  HPRBamFW primer
aaaggatccatggcgaaaccggtgtccat

SEQ ID NO: 9 - DNA, 30 bp, Artificial
  RD29AP1 primer
tttaagcttggagccatagatgcaattcaa

SEQ ID NO: 10 - DNA, 33 bp, Artificial
  RD29AP2 primer
aaatctagactttccaatagaagtaatcaaacc

SEQ ID NO: 11 - DNA, 29 bp, Artificial
  HPRXbaRE primer
aaatctagacgtttccatgtcacaggttg

SEQ ID NO: 12 - DNA, 29 bp, Artificial
  HPRSacFW primer
aaagagctcatggcgaaaccggtgtccat

SEQ ID NO: 13 - DNA, 29 bp, Artificial
  HPRSacRE primer
aaagagctccgtttccatgtcacaggttg

SEQ ID NO: 14 - DNA, 29 bp, Artificial
  HPRP1Hind primer
aaaaagcttgaagcagcagaagccttgat

SEQ ID NO: 15 - DNA, 30 bp, Artificial
  HPR2Bam primer
aaaggatcccgccatggtagagaaaagaga

SEQ ID NO: 16 - DNA, 30 bp, Artificial
  HPR3Hind primer
aaaaagcttacgtcagcattatctcgttac

SEQ ID NO: 17 - DNA, 44 bp, Artificial
  Oligonucleotide Adapter 1
ctaatacgactcactatagggctcgagcggccgcccgggcaggt

SEQ ID NO: 18 - DNA, 8 bp, Artificial
  Oligonucleotide Adapter 2
acctgccc

SEQ ID NO: 19 - DNA, 27 bp, Artificial
  AP1 primer
ggatcctaatacgactcactatagggc

SEQ ID NO: 20 - DNA, 20 bp, Artificial
  28w1 primer
agctggcgtaatagcgaaga

SEQ ID NO: 21 - DNA, 18 bp, Artificial
  AP2 primer
ctatagggctcgagcggc

SEQ ID NO: 22 - DNA, 20 bp, Artificial
  28w2 primer
cgttggagtccacgttcttt

SEQ ID NO: 23 - DNA, 30 bp, Artificial
  28LAP1 primer
gttactgctgtgtttcttgcgaggtgactc

SEQ ID NO: 24 - DNA, 32 bp, Artificial
  28LAP2 primer
ctcaaagctgagaacagagtctctccccaatc

SEQ ID NO: 25 - DNA, 20 bp, Artificial
  NPT 1 primer
attgcacgcaggttctccgg

SEQ ID NO: 26 - DNA, 19 bp, Artificial
  NPT 2 primer
atcgggagcggcgataccg

SEQ ID NO: 27 - DNA, 21 bp, Artificial
  GG9 primer
ctgcatccggcgaccttgttc

SEQ ID NO: 28 - DNA, 27 bp, Artificial
  HPRXbaFW primer
aaatctagaatggcgaaaccggtgtcc

SEQ ID NO: 29 - DNA, 28 bp, Artificial
  HPRSalRV primer
aaagtcgactcatagcttcgaaacaggc

SEQ ID NO: 30 - DNA, 6 bp, Artificial
  TATA box consensus sequence
tataat